(19) United States
(12) Patent Application Publication
(10) Pub. No.: US 2024/0130820 A1
(43) Pub. Date: Apr. 25, 2024

(54) MEDICAL CO-WORKING SPACE WITH SCHEDULING AND PROVISIONING SYSTEM

(71) Applicants: Jessica P. Wu, Los Angeles, CA (US); Florin C. Toader, Los Angeles, CA (US)

(72) Inventors: Jessica P. Wu, Los Angeles, CA (US); Florin C. Toader, Los Angeles, CA (US)

(21) Appl. No.: 18/490,709

(22) Filed: Oct. 18, 2023

Related U.S. Application Data
(60) Provisional application No. 63/380,162, filed on Oct. 19, 2022.

(51) Int. Cl. A61B 50/13; G16H 10/60; G16H 40/20; G16H 40/40

(52) U.S. Cl. CPC A61B 50/13; G16H 10/60; G16H 40/20; G16H 40/40; A61B 2562/08

(57) ABSTRACT

A medical co-working space configured to perform patient intake, including limited physiological assessment, and manage access to rooms, storage and medical products during a physician clinic schedule.

### CROSS REFERENCE TO RELATED APPLICATIONS

This application claims priority to U.S. Provisional Patent Application No. 63/380,162, filed Oct. 19, 2022, entitled “MEDICAL CO-WORKING SPACE WITH SCHEDULING AND PROVISIONING SYSTEM,” which is incorporated by reference herein, in the entirety and for all purposes.

### BACKGROUND

The embodiments herein relate generally to medical co-working spaces, and more specifically to methods and apparatus for scheduling and provisioning of medical facilities, equipment, supplies, personnel and/or services for both physicians and patients.

Traditionally physicians that lease their own offices hire real estate brokers to identify suitable space and negotiate a lease with the landlord. This process is laborious and can take from 3-12 months. The lease locks the physician in to the space from 5 to 10 years or more. After this period, the physician has the option to extend for another period (typically 5 or more years) or start the process anew. Once the lease is in place the physician has to build out the office to suit their needs. This process can take from 5-12 months.

### BRIEF SUMMARY

A medical co-working space configured to perform patient intake, including limited physiological assessment, and manage access to rooms, storage and medical products during a physician clinic schedule.

In one embodiment, a medical co-working space is provided, comprising a reception area, one or more check-in stations located in the reception area, each check-in station comprising a non-contact thermometer assembly configured to measure a patient temperature, a display, a patient input device, a security access dispenser, a plurality of medical spaces, wherein at least two of the medical spaces are patient examination rooms, each examination room comprising a remote room door control lock, a room door security access sensor, a patient examination table, a first display, a first remote storage compartment control lock, a first storage compartment, a ceiling or wall mounted camera, a scheduling and provisioning server, comprising a database of clinician profiles, a database of patient profiles, wherein each patient profile is associated with a clinician profile in the database of clinician profiles, a database of clinician medical space reservations, wherein each of the clinician medical space reservations is associated with a clinician profile, at least one patient examination room of the plurality of medical spaces, and comprises a reservation time period, a communication link to the one or more check-in stations, a verification module configured to receive data from the patient input device, the PPG assembly and the thermometer, and actuate the security access dispenser based on the received data, a security controller configured to control the remote room door control locks and the first remote storage compartment control locks based on the reservation time period and the at least one patient examination room associated with each reservation, a display server configured to display clinician information on the first display of the patient examination room during the corresponding reservation time period of a clinician medical space reservation for one of the patient examination rooms. The security access dispenser may be a code printer or a security card dispenser. The clinician information may comprise at least one of a medical school diploma, residency certificate, fellowship certificate and board certification. The clinician information may be a scanned image of a physical medical school diploma, residency certificate, fellowship certificate or board certification. The scheduling and provisioning server may be configured to generate a clinician medical space reservation for the database of clinical medical space reservations based on a reservation request associated with a clinician profile in the database of clinician profiles and the existing clinician medical space reservations in the database of clinician medical space reservations. The patient examination rooms each further comprise a first storage compartment security access sensor. The plurality of medical spaces may further comprise at least one waiting room, the at least one waiting room comprising a remote waiting room door lock, a plurality of seats, and a waiting room display. The plurality of medical spaces may further comprise at least one nursing station, at least one laboratory room, at least one patient check-out area, at least one specimen room and/or at least one waiting room. The at least one waiting room may further comprise a refreshment station. The specimen room may comprise a bathroom. The at least one specimen room further comprises a specimen cabinet with a door, wherein the specimen cabinet is located in a wall of between the specimen room and a laboratory room. Each of the remote room door control lock and the first remote storage compartment control lock may comprise a visual status indicator configured to facilitate identification of unlocked or unlockable rooms or storage compartments. The co-working space may further comprise a plurality of mobile carts, wherein each of the plurality of mobile carts comprises a cart housing, a plurality of wheels attached to the cart housing, a plurality of drawers in the cart housing, a cart door movably attached to the cart housing, the cart door comprising a closed configuration wherein the cart door is located over the plurality of drawers to resist drawer opening, and an open configuration wherein the cart door is located away from the plurality of drawers to permit drawer opening, an electronically actuatable door lock, a door lock sensor, and an RFID identifier. The cart housing of each of the plurality of mobile carts may comprise at least one wall attachment structure, and each examination room of the plurality of examination room comprises at least one cart attachment structure. The at least one wall attachment structure and the at least one cart attachment structure may comprise complementary Z-clip strips. The at least one wall attachment structure may comprises a plurality of wall attachment structures, and wherein the at least one cart attachment structure may comprise a plurality of cart attachment structures, and wherein the plurality of wall attachment structures and the plurality of cart attachment structures may each have an elongate horizontally oriented shape. The plurality of wall attachment structures and the plurality of cart attachment structures may comprise complementary horizontally staggered or offset arrangement. Each of the plurality of mobile carts further comprise a door closure sensor.

In another embodiment, a mobile cart is provided, comprising a cart housing, a plurality of wheels attached to the cart housing, a plurality of drawers in the cart housing, a cart door movably attached to the cart housing, the cart door comprising a closed configuration wherein the cart door is located over the plurality of drawers to resist drawer opening, and an open configuration wherein the cart door is located away from the plurality of drawers to permit drawer opening, a plurality of elongate Z-clips arranged in a horizontal parallel offset configuration on a rear surface of the cart housing, a plurality of surface contact connectors located on the plurality of elongate-Z-clips, and an electronically actuatable door lock connected to at least two of the plurality of surface contact connectors. The mobile cart may further comprise a door lock sensor connected to at least two of the plurality of surface contact connectors, and/or a door closure sensor connected to at least two of the plurality of surface contact connectors.

In one embodiment, a mobile cart system is provided, comprising a cart housing, a plurality of wheels attached to the cart housing, a plurality of drawers in the cart housing, a cart door movably attached to the cart housing, the cart door comprising a closed configuration wherein the cart door is located over at least some of the plurality of drawers to resist drawer opening, and an open configuration wherein the cart door is located away from the plurality of drawers to permit drawer opening, a pair of spring-biased voltage connectors that are either vertically aligned along a midline between two halves of the cart housing, or symmetrically spaced from the midline, a plurality of pairs of spring-biased surface contact connectors, wherein each pair is symmetrically spaced away from the midline, and an electronically actuatable door lock connected to at least one pair of the plurality of surface contact connectors. The mobile cart system may comprise a z-clip located symmetrically on the midline. Each pair of spring-biased surface contact connectors may comprises spring-biased z-clips. The pair of spring-biased voltage connectors may be symmetrically spaced from the midline. The pair of spring-biased voltage connectors may be spring-biased z-clips. Each pair of the plurality of pairs of spring-biased surface contact connectors may comprise spring-biased z-clips. The pair of spring-biased voltage connectors and the plurality of pairs of spring-biased surface contact connectors may be horizontally aligned. The pair of spring-biased voltage connectors and the plurality of pairs of spring-biased surface contact connectors may each comprise z-clips. The mobile cart system may further comprise a cart identification tag. The cart identification tag may be located on a rear wall of the cart housing. The cart identification tag may be located symmetrically on the midline. The cart identification tag may be spaced closer to a top edge of the cart housing than to the closest of any pair of the plurality of pairs of spring-biased surface contact connectors. The identification tag may be a QR code, NFC tag, Bluetooth beacon, or RFID tag.

The medical co-working space comprises a scheduling and provisioning system that allows multiple unaffiliated physicians to rent medical office space and see patients concurrently. The system is built to reduce or eliminate inefficiencies in the workflows and processes involved, with efficiency measured in the ease of the rental process, of the office-provisioning process from the physician's standpoint and with turnover of the medical office spaces from the management perspective, of the handling of patient flows in and out of the offices and facility.

The system allows the physician to lease as little as one room, for as little as one 3-hour time or other slot. This duration matches what physicians typically refer to as a “clinic” or half-day of “clinic”, though the system may be configured to permit smaller or larger minimum time slots. The rental reservation can be made at any time with or without any membership-fee and with or without any extended time-commitment. The medical co-working space may be custom-built its space to specifically suit physicians so that physicians are familiar with their offices, the exam and procedure rooms, the nurse's station, the waiting room and checkout area, the lab, etc.

The medical co-working space and system allows physicians to reduce their overhead by only leasing the minimum required space. If physician is only at a medical co-working space for a few hours at a time, there is no need to pay for a kitchen or for a staff restroom. If the system directs patients from check-in area to waiting room, to exam room, to checkout and exit, the physician needs fewer staff and therefore even less office space. If the system communicates to in-bound patients an updated appointment time, then these patients can wait off-site until their actual appointment time. This reduces waiting room need (not to mention increasing patient well-being and reducing their stress upon encountering their physician). These efficiencies may facilitate increased usage of medical co-working spaces versus a traditional medical office, and reduce overhead medical office costs that may improve the availability of healthcare in rural areas.

### BRIEF DESCRIPTION OF THE DRAWINGS

FIG. 1 depicts an exemplary floor plan of a medical co-working site.

FIG. 2 depicts the software architecture for the physician account module and scheduling module.

FIGS. 3A and 3B are schematic side and end elevational views of an exemplary examination room configuration.

FIGS. 3C and 3D are schematic side and end elevational views of the room configuration in FIGS. 3A and 3B without the mobile cart.

FIG. 3E is a schematic side elevational view of another exemplary examination room configuration that is a mirror configuration of the room in FIGS. 3A to 3D with the cart interface to the right of the exam room door.

FIG. 4 is a schematic diagram of the provisioning module and exemplary connected hardware.

FIG. 5 is a schematic diagram of the software modules of the orchestration server and the hardware interface.

FIG. 6 is a schematic diagram of an exemplary hardware configuration and local orchestration server.

FIG. 7 is a schematic diagram of an exemplary cloud-based central network system for a plurality of co-working facilities.

FIG. 8 is a schematic dataflow diagram between a central server, remote site-specific server and local server.

FIG. 9 depicts an exemplary software control process for performing initial patient arrival at the co-working site.

FIG. 10 depicts an exemplary software control process for unscheduled patient arrival.

FIG. 11 depicts an exemplary software room provisioning and schedule completion process.

FIGS. 12A and 12B are schematic front perspective views of an exemplary mobile cart in a closed and open configuration, respectively.

FIG. 12C is a rear perspective view of the mobile cart in FIGS. 12A and 12B, depicting the cart interface.

FIG. 12D is a schematic front perspective view of the mobile cart in FIG. 12B without the drawers and depicting the schematic wiring configuration.

FIG. 12E is a schematic electrical diagram of the complementary cart and wall interfaces.

FIG. 13A is a schematic frontal view of exemplary engagement structures of the cart and wall interfaces.

FIG. 13B is a schematic side view of the engagement structures in FIG. 13A.

FIG. 13C is a schematic side of the engagement structures in FIGS. 13A and 13B in a separated configuration.

FIGS. 14A and 14B are schematic front and side cross-sectional views, respectively, of another variation of an exemplary mobile cart with a partial cover.

FIGS. 14C and 14D depict the mobile cart of FIGS. 14A and 14B on an uneven floor and against an uneven wall, respectively.

FIGS. 14E and 14F schematically illustrate the mobile cart of FIGS. 14A and 14B spaced apart and against a patched taped wall, respectively.

FIGS. 15A and 15B depicts another exemplary mobile cart with side and back connectors being used in rooms with different orientations of docking locations.

FIGS. 16A and 16B depict the exemplary mobile cart in FIGS. 15A and 15B before and after inadvertent bumping, respectively.

FIG. 17A is a close-up side schematic side view of an exemplary mobile cart and corresponding wall with a spring-mounted z-clip.

FIG. 17B is a schematic side view of an exemplary mobile cart and corresponding wall with two z-clip assemblies.

FIG. 18 is a schematic close-up side schematic view of an exemplary mobile cart with a z-clip assembly and a contact plate assembly.

FIG. 19 is a schematic rear view of a cart with a passive z-clip anchor and a plurality of electrical connectors.

FIGS. 20A and 20B are schematic rear views of a mobile cart with a plurality of z-clip electrical connectors, with and without an identification tag, respectively.

FIGS. 20C to 20F depict additional variations of horizontally aligned connector configurations.

FIGS. 21A to 21D are plan, front, rear and side views, respectively, of another exemplary mobile cart with midline voltage connectors.

FIGS. 22A to 22D are schematic rear views of exemplary mobile cart connector configurations with voltage connectors along a vertical midline.

FIGS. 23A and 23B are schematic rear views of additional exemplary mobile cart connector configurations with signal connectors spaced apart horizontally and vertically, respectively.

FIG. 24 is a schematic state machine diagram to manage the locking and unlocking of a remote lock by the lock controller.

FIG. 25 depicts a database organization of a user account for ordering a mobile cart or office.

FIG. 26 is a room map illustrating the use of resource activation to refine geo-location of an object or person.

### DETAILED DESCRIPTION

### Overview

The scheduling and provisioning system allows physicians to rent available medical space in its facilities through its website. The rental lease process can be completed in between 5-10 minutes. This process includes an initial account setup followed by a selection of day(s) and hour(s) needed along with the type of medical space and any ancillary room(s) needed. The process completes with the physician provisioning the selected space(s), and purchasing any other service(s) and/or products needed. Payment is taken upon checkout.

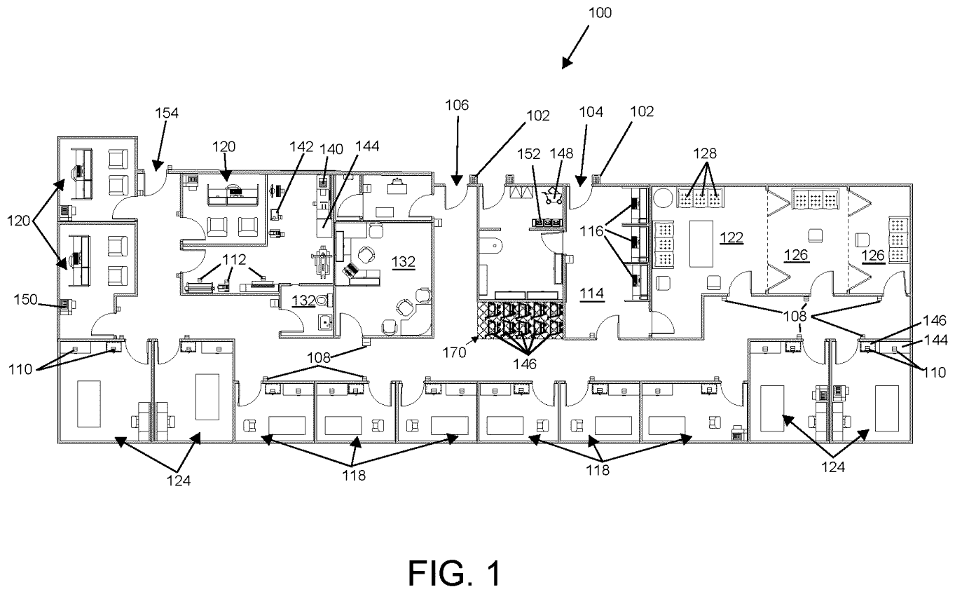

Referring to FIG. 1, on the selected date/time the physician enters the medical co-working site 100 and after verifying their access to the site 100 via an entrance security interface 102 at the physician entrance 104 or patient entrance 106. In other variations, a shared entrance may be provided. The physician is given a ticket or other security access device that grants access to all the rooms/services/products purchased via electronic locks 108, 110, 112 with RFID scanners, optical scanners, and/or biometric scanners, and the like. Attempts to access anything not rented/purchased are denied.

The physician's patients are also given tickets or other security access devices upon at the patient entrance 106 to the foyer or check-in room 114 of the medical co-working site 100. Depending on the room(s)/option(s) purchased by their physician and whether further patient check-in is required by the patient at a check-in station 116 in the check-in room 114, each patient is directed by the scheduling and provisioning system to their designated location(s), such as an examination room 118, consultation room 120, or waiting room 122, or procedure room 124. In other variations, patients may perform remote-check-in via a mobile app or desktop application, and so use of the onsite check-in stations 116 is not utilized. Attempts to access anything not rented/purchased by their admitting physician are denied. The entrance security interface 102 may include a display and one or more input assemblies, e.g. keyboard or keypad, touchscreen overlay, optical scanner, RFID/NFC reader and the like to facilitate data input, and optionally a security code dispenser, e.g. ticket printer or RFID fob dispenser. In other variations, a code dispenser is not required, as the security system is able to provide user access via a mobile application or via cameras with facial recognition at the security control locations and access points.

The physician is notified of the patient and their location and proceeds with their examination, procedure or healthcare visit. Once the physician completes its examination, the system directs patients to the facility's exit 154 and marks the patient's ticket as no longer usable. The exit may be the same as the physician 104 or patient entrance 106.

The process repeats for the duration of the physician's rental period. At the end of the rental period, the physician exits the site and the scheduling and provisioning system marks their ticket or security device as no longer usable. Depending upon the options purchased, the system determines if there are any outstanding charges that need to be settled and, if so, deducts them from the rental deposit taken at initial checkout. If there are no such charges, the system refunds the deposit to the physician and the lease is completed.

### Account Creation

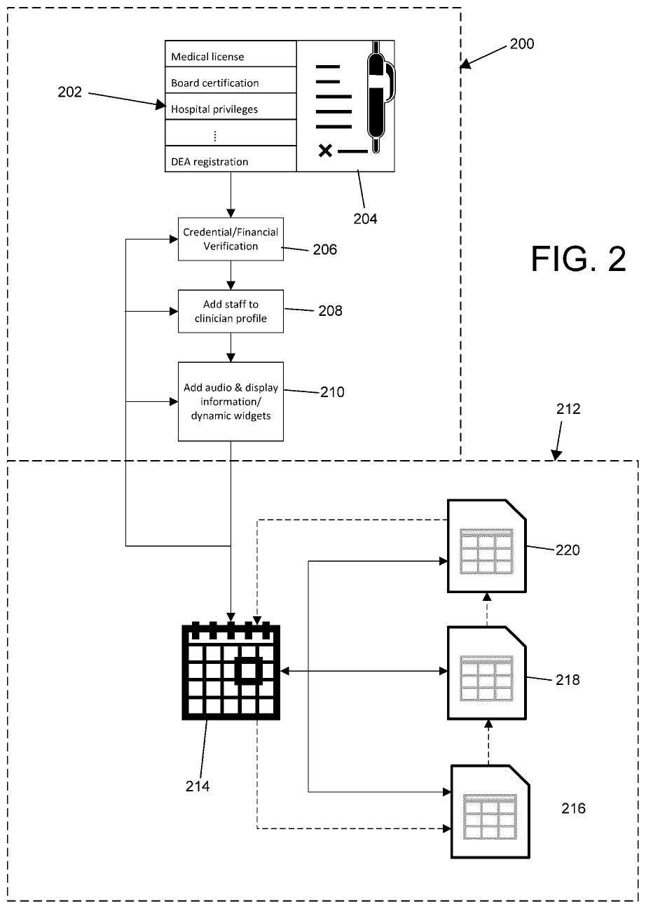

A physician or other clinician looking to lease space at the medical co-working facility for the first time may preferably visit the website and create an account. Alternatively, the physician can schedule a visit to the facility and complete the account registration onsite. Referring to FIG. 2, the physician account module 200 utilizes a web-based, mobile or desktop application to perform initial account creation and to enter general contact information, financial information and logon credentials, as well as healthcare credentialing information 202, such as medical license, specialty, any hospital and professional affiliation(s), driver's license, National Provider Identifier (NPI) and/or DEA information, along with images of these documents. The physician account module 200 also includes disclosure of the lease terms and electronic signature of the lease contract 204. After completion of a verification process 206 of the credentialing and other information, and any other verification is performed, e.g. credit check, criminal background check, the registration is completed. The verification process 206 may be automated via field matching of entered information based on public and/or proprietary healthcare and insurance databases associated with the physician information, but in some variations may include manual verification. The clinician may now proceed to customize their account or move directly to the rental process. The customization can be done at any time after the basic account has been created. Periodically, the account verification process may be repeated based on license expiration dates or medical board regulatory action reports.

### Account Customization

The physician account module 200 allows the physician to add staff 208 to their account, such as nurses, receptionists, nurse anesthetists, nurse practitioners, medical assistants and/or physician assistants. Once added to the account, the physician can assign one or more of the staff to a given lease, and optionally to assign full or partial account rights to one or more staff. In some variations, the staff may be permitted to enter the co-working facility without the physician being present. In some variations, the staff are permitted to access and work under the physician account without further credential verification, while in other variations of the system, licensed staff undergo a corresponding credential verification process similar to those of the physician, before they become active under the physician account or profile.

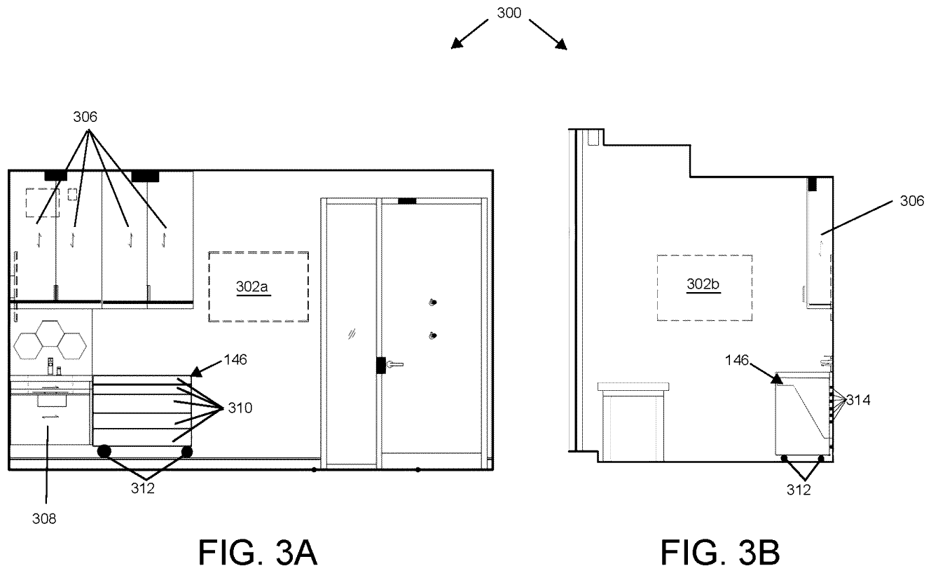

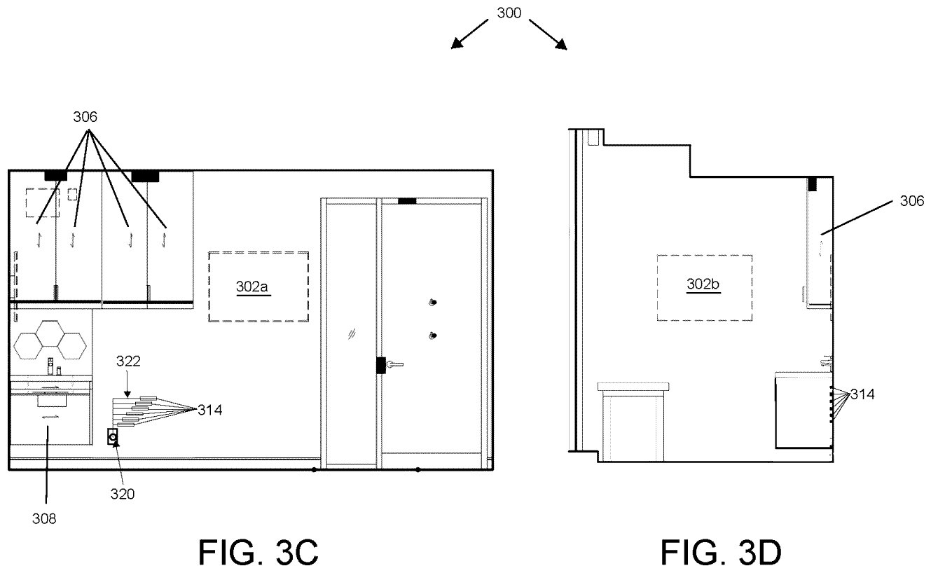

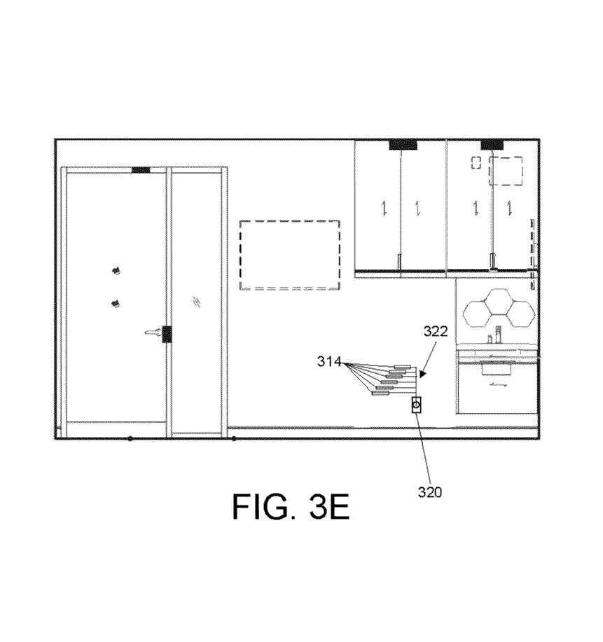

In some variations, the reservable offices, procedure rooms or examination rooms may have one or more digital displays or screens mounted in the room or on a rollable media cart. In one example depicted in FIGS. 3A and 3B, main reservable room type configured as an examination room 300 has two digital screens 302a, 302b, one screen 302a mounted on one wall 304a and the second screen 302 on a different wall 304b. These screens 302a, 302b can display any digital collateral for the duration of a clinic reservation time period and can be customized by the physician with information or other products located in their profile through the display customization interface 210 physician account module 200 in FIG. 2. The physician can choose their own images to be displayed. Images, such as school diplomas, medical school certificates, professional proof of qualification, or other personal memorabilia can be uploaded and then specified to shown in desired room(s) and screen(s). In further variations, medical and wellness advertisements may be provided on one or more screens. This content may be selected by the physician or clinician, or the physician or clinician may elect for an advertising module in the scheduling and provisioning system to select the advertisements based on the physician or clinician clinical certification(s). A variety of other dynamic or interactive digital objects or widgets may also be displayed, e.g. weather, wait time, disease information, traffic information, news, etc. In some further variations, the configuration of the initial screens 302a, 302b and selection of displayed information may be performed or updated may also be performed through the screens themselves in the examination room 300 if a touchscreen interface is provided on the screens 302a, 302b, or the physician mobile app. Some of the objects or widgets may be patient-selectable while awaiting the physician, or may be physician-selectable during the patient visit, e.g. depicting patient lab results or imaging.

In some variations, the physician may also select music or music service to be played in each of the leased rooms. Styles such as jazz, easy listening, classic, etc. can be selected and specified for different rooms. In some variations, the scheduling module 212 associated the physician account may store and use streaming service account information entered by the physician, or to offer commercial licenses for such services on a per session or clinic basis. In some variations, the patient may be able to override the default selection.

Once these choices have been uploaded and configured, unless the physician makes different or overriding selections, the system may be configured to automatically configure rooms with the same options, or to otherwise pre-populate the room configuration user interface for physician confirmation.

### Lease Process

Once a physician account is established, the scheduling module 212 may be activated or selected so that the physician can select via a scheduling interface 214 one or more available time slots, e.g. a three-hour period, on an available date, and for an available room type, or to join a waitlist for an unavailable selection. As noted previously, the scheduling module 212 may default to or limit reservations to a minimum three-hour time slot because this time allotment, traditionally termed a “Clinic”, as widely used in the medical field and therefore familiar to all physicians. Morning, afternoon and evening time slots may be provided, as well as bundled or repeating periodic time slots.

The scheduling module 212 shows the physician what Clinics are still available to rent using a calendar-based user interface (UI) 214. For each Clinic the system proceeds to display the main types of rooms still available for lease (e.g. Exam Room 118, Physician's Consultation Office 120, Procedure Room 124, Psychiatrist/Psychologist's Office 126 in FIG. 1)—what the scheduling module 212 may refer to as the available room inventory.

Upon making a Date/Clinic Time/Room selection, the scheduling module 212 may present an ancillary space interface 216 to optionally reserve any available ancillary space types that may be rented: a seat 128 in the Waiting Room 122, the Nurse's Station 130, the Specimen room 132 (akin to a medical rest room that may or may not be configured to accept a biological sample through an access compartment 134 that may be in communication with a Laboratory 136 adjacent to the Specimen Room 132. These are ancillary room types because they can only be rented in conjunction with the rental of a main room type, though in some other variations, the Laboratory may be leasable without a main room type reservation.

Next, another user interface 218 may be presented so that the user can optionally choose one or more ancillary product(s) that are available to rent. Ancillary rentable products are products that can only be rented in conjunction with the rental of a main room type. These products may include of auto-clave 140 to sterilize medical devices, centrifuge to spin blood samples, refrigerator 142 to store medical products, immobile locker(s) 144 for storing personal belongings during one or across multiple clinics, mobile cart(s) 146 for storing personal medical supplies during one or multiple clinics, laboratory pickup 148 of patient(s)'s samples, or a printer 150. And depending on the physician's past rental history and rental volume, the physician could also qualify to rent a mailbox 152.

Last, the user may also be shown an interface 220 with any available optional services for purchase in the room selected: disposal of sharp objects, cleaning of room by facility staff in lieu of physician's own staff. Select medical supplies available for purchase are also presented at this time: medical trays, medical tray liners, patient gowns, surgical disinfectant wipes, test tubes, specimen sample containers, bandages, etc. Although these interfaces 216, 218, 220 are depicted as directly accessible from main interface 214 of the scheduling module 212, the interfaces may be presented serially (as depicted by the dashed lines) during the initial leasing process

As each of these selections are made, the system displays the running total, along with the additional deposit needed, on the website, or mobile/desktop application.

The process is repeatable for as many Dates/Clinics the physician wishes to lease. Before checking out the physician is allowed to add one or more of their staff to the lease.

This completes the on-boarding process and the physician is presented with the amounts due for the rental/purchases and the corresponding rental deposit. The physician checks out using their credit card or direct deposit. In some variations, first-party or third-party financing may be available. Upon successful payment, the physician and any staff added to the lease, are emailed or issued an alphanumeric code along with a corresponding QR or bar code which will allow entry to the site at the time of the leased clinic(s). Alternatively, a pre-existing RFID fob or smartphone NFC feature may be activated in the system and used to provide site entry and access to other unlockable locations and features of the medical co-working space.

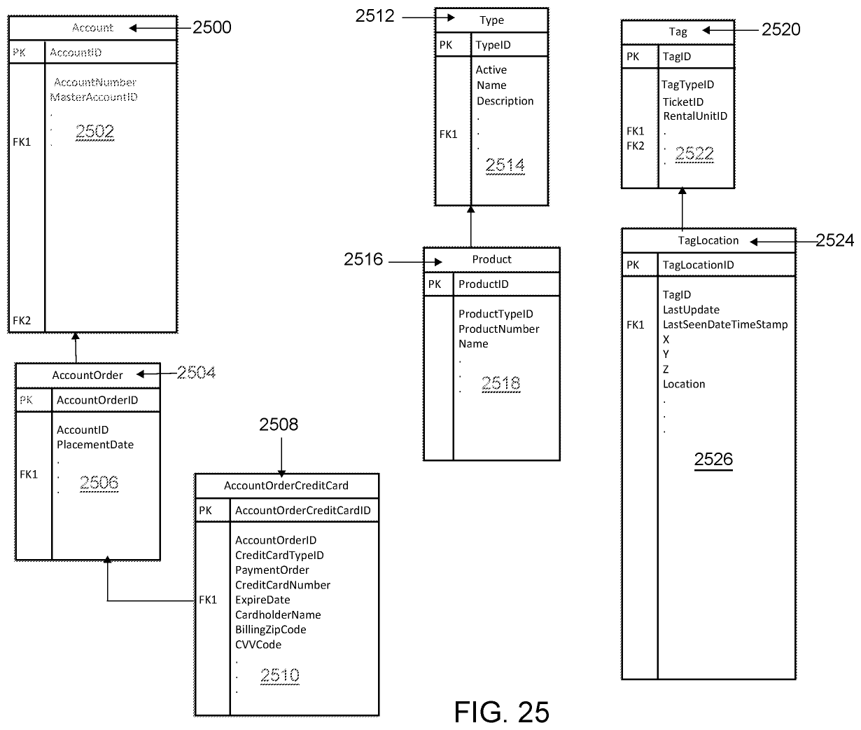

Referring to FIG. 25, during a typical leasing order, a cart and an exam room may be reserved for use. An account 2500 comprising master physician information elements 2502, including financial information may be selected and confirmed, and an account order 2504 with order elements 2506 such as order date and associated physician account information is generated. The account order 2504 will further generate a credit card charge 2508 with billing elements 2510, account order and master account associated with it. Each leasable item, e.g. mobile cart and examination room, has an associated resource type 2512 and a group of associated product class information 2514. A unique product specific profile 2514 is also associated with each resource type 2512, including serial numbers acquisition date 2518, as well as a geo-location tag identifier profile 2520. The tag profile 2520 includes specific tag ID elements 2522, and a tag location profile 2526, which contains any of a variety of location-related information 2528 for the identifier tag, which may include raw GPS signals, estimated location components and associated probability of accuracy, timestamp information, etc.

### Lease Customization

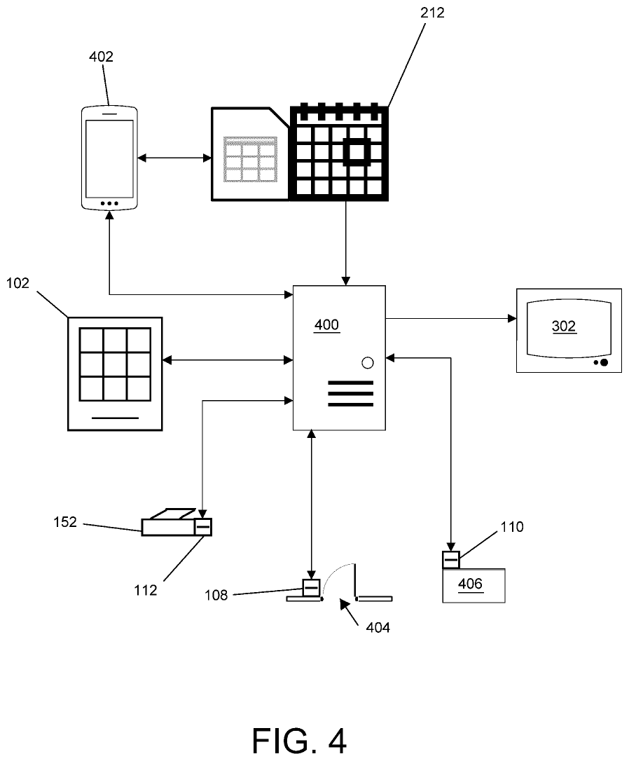

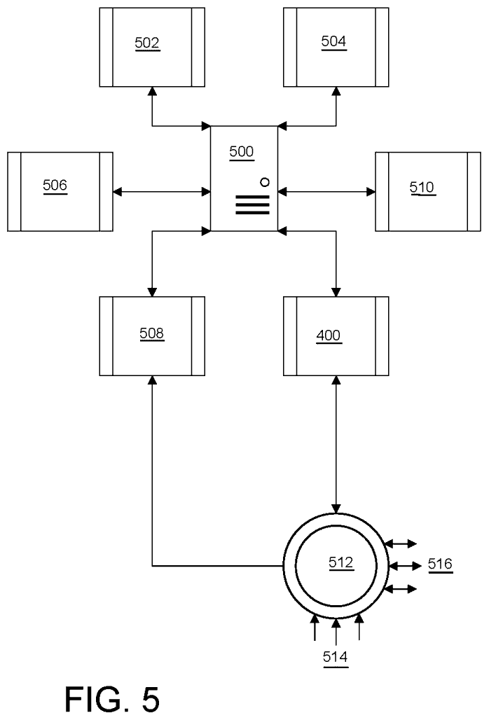

Each exam room, procedure room or other leasable room may be provided with a remote controlled room lock 108 of a door 404 which is controlled by the provisioning module 400, depicted in FIG. 4. The provisioning module 400 may also manage the lock/unlock status of the entrance locks 102. Within the rooms, fixed cabinets 406 with doors may be provided that have also remote controlled storage locks 110 controlled by the provisioning module 400. Each of the remote controlled locks 108, 110 may include a visual indicator, such as a LCD or LED screen, RGB lights, to indicate the state of each lock 108, 110, whether locked, unlocked or unlockable, or out-of-order, for example. A code scanner may also be provided with the lock to require detection of a particular security code before it is unlocked. Alternatively, the provisioning module 400, which is part of the orchestration server 500 depicted in FIG. 5, may utilize the geo-location information of a physician, staff or patient received from cameras or RFID sensors to and actuate a lock 108, 110 based on detected proximity of an authorized person. This, for example, can allow the opening of a cabinet during a clinic visit by an authorized physician or staff person, while blocking access by a patient or unauthorized physician or staff person. Referring back to FIG. 4, the provisioning module 400 also manages the remote controlled equipment locks 112 of any ancillary equipment, based on scheduling information of the physician and patient in the scheduling module 212, or from real-time physician or manager applications 402. In some variations, the equipment locks 112 for equipment like the printer 152 may be network controlled, rather than a physical lock. The provisioning module 400 through the orchestration server may also be configured to output desired information onto the displays 302 through data received from the physician account module. Examples of such locks include those by VON DUPRIN (Indianapolis, IN), HID (Austin, TX), ATOPLEE (Shenzhen, China) and LEVITON (Melville, NY). In addition to the remote door or cabinet locks, door sensors to detect door status may be provided, including but not limited Reed sensors, e.g. QWORK (Stockholm, Sweden, GE (Boston, MA).

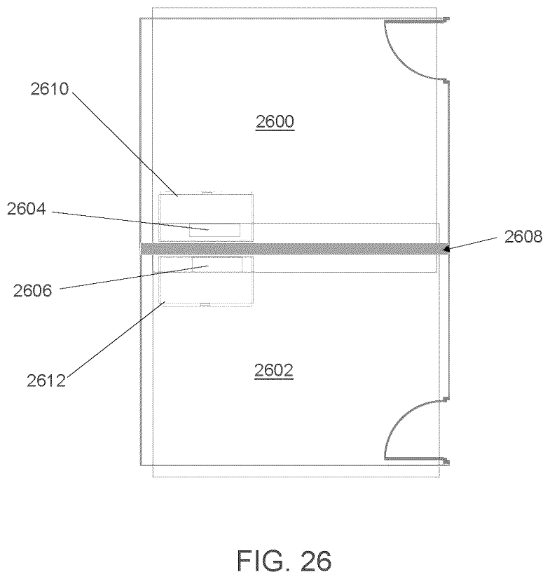

Geo-location may be obtained using signal strength from a plurality of proximity sensors a probabilistic algorithm, e.g. clustering, threshold, template fit, curve model algorithm to estimate the person or object location. The estimated location may be further refined based on the user's resource activation, e.g. resolving the location of a patient or physician based on the physician's activation list or map of resources activated or leased during the work period. Various known geo-location technologies have a limited resolution that may vary depending on the setting or presence of interference. GPS, for example, has a minimum resolution of 2 feet when in an outdoor setting that is unobstructed from the sky. The presence of buildings, however, may reduce resolution to as low as 16 feet, or even worse when indoors. The detection profile of other geo-location technologies may vary. For example, Near Field Communication (NFC) tags have a high resolution in inches but has an extremely limited range of only a few inches, and thus cannot be used to detect a cart location within a building except when docked at a specific location with an NFC reader. FIG. 26, for example, depicts two adjacent rooms 2600 and 2602 each containing an antenna or proximity sensor 2604, 2606 using GPS, WiFi and/or RF to identify geo-location of an object or person, and are separated by a wall 2608, e.g. 5 inches thick, and each containing a cart 2610 and 2612 with an identification tag. Due to the proximity of each sensor 2604, 2606 to each other, each sensor 2604, 2606 may detect both carts 2610, 2612. The wall 2608 may not consistently attenuate the signal of the farther cart, and so the sensor 2604, 2606 may not consistently, reliably or accurately detect which cart is located in the corresponding room 2600, 2602. This may be the result of multi-path signals, where metal in walls (e.g. studs) or rebar in concrete floor may act as antennas and amplify or weaken signals, such that farther objects or persons may have stronger geo-location signals detected than closer objects or persons. To help improve the accuracy of geo-location or to resolve some ambiguity in the geo-location estimate, non-sensor information relating to the user's leasing information may be utilized. For example, increased weighting or altered thresholding of signals may be provided to sensors, or corresponding to rental location or other rental attributes may be used to supplement the geo-location. For example, if a physician leased room 2602 but not room 2600, this rental information may be used to reject sensor information from sensor 2604 if the corresponding room 2600 was not leased.

In some variations, a consistent inventory arrangement or stocking scheme is provided throughout the rooms or room types, so that the clinicians and staff can develop a familiarity with the co-working space regardless of the assigned room or facility. For example, referring back to FIG. 3, cabinets 306 mounted at eye level or above may be used for storing site inventory. Cabinets 308 mounted at counter-top level and below may be configured to contain supplies that can be purchased and used by physicians. Cabinets in office rooms may be used to store site inventory. The provisioning module 400 may utilize RFID tags affixed to product stock, weight sensor arrays and/or image analysis in order to inventory the contents of these cabinets in real time. Physicians may purchase these products beforehand at a reduced price; they can also avail themselves of these products during their clinic, without having purchased them during the lease process. In some variations, however, the physician will be charged a higher price with items purchased on-demand, compared to pre-purchased or pre-ordered items.

The orchestration server 500 may also be configured to allow the physician to specify any rented rooms they do not want the system to use for seating patients, i.e., a physician or staff person might not want patients to be seated in their rented physician's office—only in any rented exam or procedure room(s). Based on any specified exclusions, the patient's ticket or security device may be configured to provide different directions to direct them to a different location within the facility 100.

The physician may also be provided several options to manage the patients who arrive outside their appointment time, and those who arrive on-time but must wait due to the physician's unavailability or who arrive accompanied by one or more companions.

The provisioning module 400 allows the physician to define an early-arrival cut-off time before which patients are automatically turned away (but asked to return at their appointment time) and after which they are accepted in the medical co-working space. The system also allows the physician to define a late-arrival cut-off time after which patients are automatically turned away from the site and asked to reschedule their appointment—patients which arrive after their appointment time but before the late-arrival cut-off time would still be allowed into medical co-working space.

Next, the physician is allowed to select a set of rules that determine how the system manages the physician's patients which arrive outside of their appointment time but within the above windows. The options may include, for example:
1) The system may be configured turn away any patients that arrive outside their exact appointment time slot. Early arrivals directed to exit the reception area and asked to return at their appointment time. Late arrivals may be directed to exit the reception area and asked to contact their physician and reschedule their appointment, or to offer a pre-selected rescheduled appointment time.
2) The system may be configured to allow any patient entry, provided there is any available space as part of the physician's lease, even if this might result in others of the physician's patient(s) being denied entry further down the schedule.
3) The physician or authorized staff member may purchase an option for extra capacity in the waiting room at a fixed price ahead of their lease time, up to a maximum number of extra seats, based on the physician's lease and availability.
4) The physician or authorized staff member may purchase extra waiting room seats during lease time, as needed if available. Each seat may be priced the same as that day's cost for a procedure room, based on availability, with a maximum dollar cap.
5) Have the system direct the patient to exit the site and building and contact their physician before leaving the area.
6) Direct the patient to enter without their companion(s).

If the physician has chosen the ancillary laboratory pickup product option, the physician or staff member will be prompted to enter the name and contact information of the courier or laboratory company. Similarly, if the physician has chosen the sharps-disposal service, they are required to enter the name and contact information of the company. Lastly, if the physician has rented a mailbox, they have the option to inform the system that they are expecting a delivery. The physician or staff member may be prompted to select the name of the delivery company. In all these scenarios, when the kiosk application makes a match between the physician's name and the delivery company, the system will assign and issue a temporary code and RFID ticket or other security access to the delivery person. The scheduling and provisioning system will instruct the delivery person to proceed to the mail room/pickup area through the door located behind them and on the right. The system will unlock the door and then relock it behind them. The system will then update the mail room/pick up area's occupancy.

Once the physician has completed the lease customization, they have the option of managing their patient flow themselves or alternatively allowing the system to do so. The physician or staff member do the latter by entering in the system, minimally, the patient phone number, patient's communication preference (voice or text), patient last name and patient's appointment time slot (anticipated start and end of the appointment), and whether the patient should be exempted from temperature check and enforcement. Without this last selection, the system would prevent a patient with temperature above normal from entering the site.

If the system is selected as the patient flow manager, the system will communicate appointment information at several times through the appointment process: once the schedule data has been entered, a day before the appointment, the day of the appointment before clinic start, and before the patient's appointment time—to appraise the patient and their physician of each other's timeliness, if the physician or patient are early/on time/behind schedule, for example.

Upon confirmation of the schedule data, the system may be configured to text, email or calls the first patient (depending on the physician's choice) and gives them basic appointment information: the date and time of the appointment, the address of the medical co-working site, and instructions to check-in once the patient is parked outside the office and ready to see the physician. They are also asked to confirm their choice of text or voice interaction with system. Schedule confirmation may be configured 24 hours, 48 hours, 72 hours or whatever time frame as set by the physician or by the system.

A day before the appointment, the patient is resent the information above and asked to confirm that they are still attending their scheduled appointment. They are reminded that they must check-in outside the office and that they cannot present themselves at the appointment without having first checked in outside.

The morning of the appointment, the patient is resent the information and asked to reconfirm their appointment. Again they are reminded they must check-in outside the building and must wait there until they are asked to come up.

If the patient has not checked-in from the parking area before their appointment time but within the arrival window defined by their physician, the system contacts the patient and reminds them of their appointment time; the system also ask them if they need to cancel their appointment.

If or once the patient checks-in from their parked car, the system will check that a physician or their staff is on site. If they are not, the system will ask the patient not to come up to the site yet; the system will update the patients 10 minutes later or once the physician or their staff have arrived. In other variations, other time periods may be used for the updates.

Once the physician and/or staff arrive, the system informs the patient they can proceed to the reception area. The system sends the patient a site access code for the reception door entrance and asks them to proceed to inside.

Should the physician opt out of using the scheduling and provisioning system to manage the patient flow, they physician or staff will then be responsible for their own patients' flow. The physician will need to communicate patient appointment time(s) and the access code to be entered at the entry kiosk; the kiosk will still dispense the RFID ticket or otherwise issue security credential to the patient as this is pre-requisite for the patient to be granted access to the site (and must be carried on the patient at all times).

The physician will then need to use the system to assign the patient to one of the physician's rented room(s), waiting room seat(s) or the generic “parking area” outside the building. From there, the physician will next need to use the system to “move” the patient to either an exam room (if they were previously in the waiting room) or to the exit.

### Scheduled General Check-In

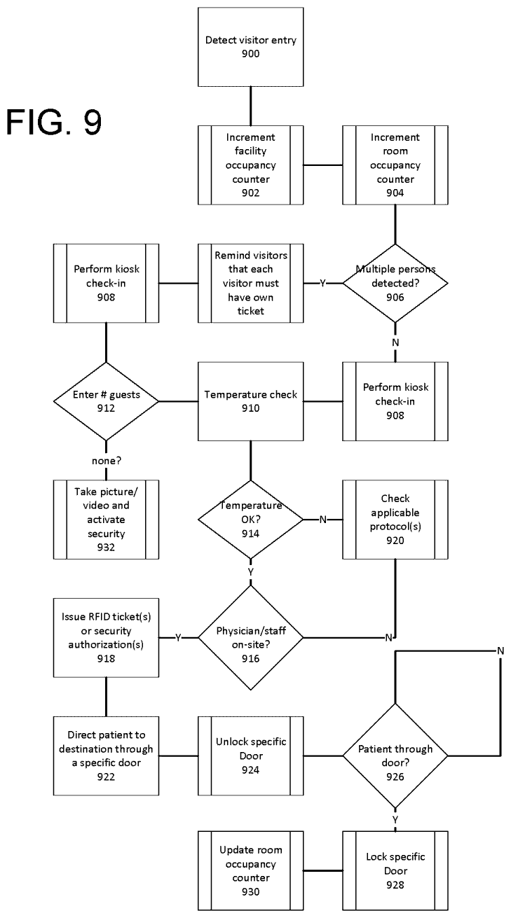

In one variation, a person arriving 900 at the medical co-working facility within the scheduled arrival window of their appointment time will experience the following workflow, depicted in FIG. 9. As they enter the facility, an electronic people counter detects them and updates the site's occupancy number 902, e.g. computer visioning counting systems by Hikvision (Hangzhou, China). This may be performed using one or more cameras inside the reception area and open source face detection algorithms. This can be one, two or more people depending on whether the patient arrived alone or accompanied by a family member, assistant or child. The system also updates the occupancy of the reception area where the persons are currently located 904.

Once in the foyer the person is greeted by one or more check-in kiosks. At the kiosks there may be a display welcoming visitors. The display updates to greet the person and directs them to use the kiosk(s) and enter their code by either typing it on the kiosk's keypad or by using the kiosk's QR code scanner. The kiosk may include an RFID sensor to confirm which patient is at which kiosk. If the system detected more than one person has entered 906, the display shows a reminder message that all facility visitors must be issued their own ticket.

At any kiosk 908 the person has the option of typing in the code communicated to them previously, or scanning the corresponding QR code. As they do either, the kiosk also takes the temperature of their hand 910. The code entered is validated and the person's temperature is checked for a normal range. If the system detected multiple patients in the foyer, it prompts the patient to enter the number of people accompanying the patient 912. If the temperature check is passed 914, the system checks that the patient's physician (or their physician's staff) is on site 916. If either one is on the premises, the kiosk dispenses the number of corresponding RFID tickets 918 (for all the visitors in the party) printed with the visitor's name and appointment information, along with their physician's name and contact phone number. If the temperature check is outside of the permitted range and/or if the physician or staff member is not on premises, the system may direct or divert the patient to a holding area as set up in the physician account. The tablet in the kiosk welcomes the visitor and directs them to the room assigned to them (by their lease if they are a physician, or by the system or their physician if the latter chose to override the system). They are directed to go to their destination using a specific door 922 which the system unlocks for them: to the waiting room using the door on the left, or to an exam/procedure/office room using the door on the right. After detecting 926 that the visitor passes through the specific dorm, the system relocks this door behind them 928. The system updates the occupancy status of the foyer 930.

If the visitor is not exempted from the temperature check, the system checks the site's pandemic protocol 920 in effect within the site's jurisdiction and proceeds accordingly: for physicians, the system denies entry if in lock-down status, allows entry with self-certification and masking-required (photo taken for verification) if in restricted status, or allows entry with warning message if there are no restrictions in place. For patients, if in lock-down or restricted status, the system denies entry if not exempted by their physician in the system, or allows entry with self-certification, warning message and masking-required in the case when there are no restrictions in the given jurisdiction.

If the visitor is a patient and is denied entry, the kiosk tablet directs them to contact their physician for instructions; the patient is also informed that they are unable to enter and directed to exit the facility until their physician directs them to return. The system proceeds to alert the physician of the situation. After detecting the patient's exit the system updates the occupancy status of the foyer as well as of the site itself. Similarly, if the physician or physician staff member is not present, the kiosk will also direct them to contact their physician for instructions or rescheduling, depending on the protocol 920 or workflow set up in the physician account.

As noted previously, if upon entry, the system detected multiple persons 906 having entered, it uses the display to instruct visitors to obtain a ticket for each person 912. If, however, the patient states they are unaccompanied then the system asks for confirmation, uses the foyer camera takes picture of the foyer and its occupants, flags the exception and alerts the Facility Manager 932.

### Unscheduled General Check-In

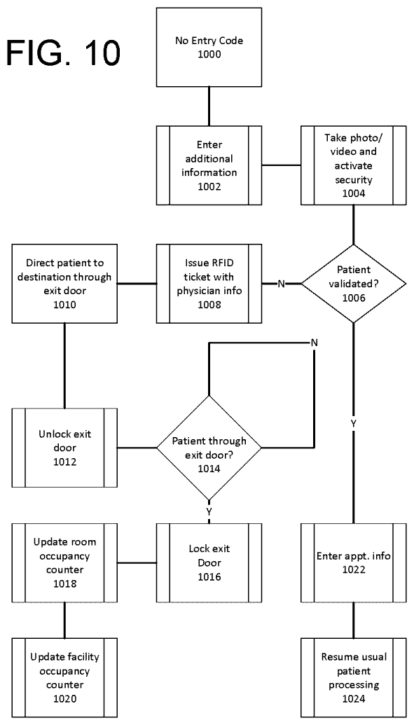

Referring to FIG. 10, if the person does not have an entry code 1000, they are asked to select the reason for their visit (Scheduled patient who has not received code/Mail/package delivery, sharps pickup, laboratory pickup, and inventory drop-off) and enter their name, phone number, company, and physician they are seeing/servicing. At the same time the foyer camera takes picture(s) of the foyer and its occupant(s), flags the exception and alerts the Facility Manager 1004. If they are validated as a patient 1006, the kiosk dispenses an RFID ticket printed with the visitor's name and appointment information, along with their physician's name and contact phone number 1008. The kiosk tablet instructs the visitor to exit the site through an exit door 1010. The system unlocks the door 1012 and after detecting that the visitor passes through the door 1014 proceeds to lock it behind them 1016. The system updates the foyer's occupancy number 1018 and facility occupancy number 1020.

If they are validated as a patient, the system performs patient validation by asking the patient to enter their last name, their phone number, their physician's name and they appointment time 1022, and resumes the usual patient processing workflow 1024.

### First-Time Clinician Check-In

When a first-time physician arrives at the co-working site at the start of their lease time. The system matches the physician's arrival time with their lease time slot and determines that the physician is entitled to entry. The system records the physician's arrival time.

The code entered by the physician is validated and the kiosk dispenses an RFID ticket printed with the physician's name and summary of leased inventory and any purchased options. The tablet in the kiosk welcomes the physician, displays their leased room(s) and reminds the physician that the RFID ticket must be carried on their person at all times.

The tablet then directs the physician to proceed using the door located in front of them and to the right. The system unlocks this door and after the physician passes through it proceeds to lock it behind them. After this message, the display changes into a directory that displays the name, specialty and main rooms of the physician. As more physicians check-in this directory updates itself to reflect the physicians currently on site.

### First-Time Clinician Staff Check-In

A staff member arrives at the co-working site at the start of their physician's lease time. The code entered by a physician's staff is validated. The system matches the staff's arrival time with their physician's lease time slot and determines that the staff is entitled to entry. The system checks to ensure that the corresponding physician is already on site. If the physician is on site then the kiosk dispenses an RFID ticket printed with the staff's name and summary of leased inventory and any purchased options. The tablet or other computing device in the kiosk welcomes the staff, displays their leased room(s) and reminds the staff that the RFID ticket must be carried on their person at all times.

The system records the staff's arrival time.

The computing device then directs the staff to proceed using the door located in front of them and to the right. The system unlocks this door and after the staff passes through it proceeds to lock it behind them.

If the corresponding physician is not yet on site, the system may be configured so that the staff is still allowed on site but the system tracks this discrepancy—to ensure that physicians do in fact present themselves physically, at some point during their lease.

### Initial Provisioning and Reservation Close-Out

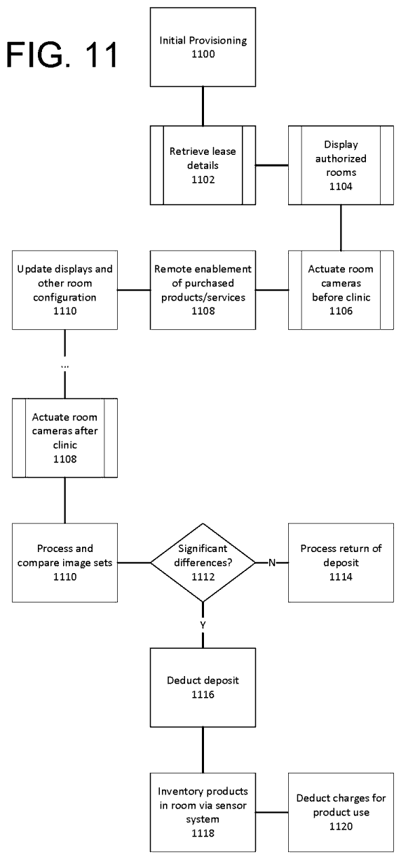

Referring to FIG. 11, having validated the physician and obtained the specifics of their lease, the system begins the initial provisioning workflow 1100 by updates the tablets outside rented rooms to display the physician's name, the room number, the unoccupied status of the room 1104. This may be optionally provided on a green background of the tablet. Doors to non-leased inventory remain locked and their corresponding tablets show directions to rooms that are leased by the physician.

The cameras in the room(s) rented change from their stowed status and take pictures or videos 1106 of the condition of the rooms: snapshots of the floors, walls, countertops, exam chairs and tables, digital displays, rolling carts, shades, and cabinets are taken and stored. These images are used to document the condition of the rented inventory prior to the physician's occupancy.

The system next proceeds to enable all the inventory purchased 1108: exam/procedure/office room door(s) are unlocked, office printer(s) turned on, and physician's movable cart(s) that is/are located in rented room(s) are also unlocked. If the physician has purchased sharps disposal service, the system unlocks the sharps container.

The displays in the rented rooms are enabled and any images configured in the physician's account are displayed as specified. The music selected by the physician in their account is played as well. The occupancy of rented room(s) is reset.

At the end of the clinic, an second series of images or video are taken 1108 and together these sets will be processes and compared 1100 by the system 1112 to the first series of images to determine 1112 automatically if any cleaning or damage fees need to be deducted from the physician's rent deposit 1116, or if manual assessment should be performed or if the room is sufficient restored to automatically process the return of the deposit 1114. Because the room cameras may be configured to provide identical framing for each series of images, simple pixel-by-pixel comparisons may be performed, to identify the total number of different pixels and/or calculating the number of pixels that exceed a certain color threshold, which may be indicative of biohazardous material still present in the room. In other variations, however, other imaging processing algorithms, including Fourier transforms, segmentation algorithms, texture analysis and other image processing algorithms known in the part may be used to initially process the image sets or videos prior to comparison. Various machine learning algorithms may also be used to perform the comparison, e.g. convolutional neuro networks which may be trained using training images from the rooms of the co-working facility itself. Should significant damage or image differences beyond one or more threshold levels be detected the system will flag it and alert the Facility Manager for special processing. The same imaging processing may be used to assess the accuracy of pre-clinic cleaning and room preparation by the cleaning staff or facility employees, compared to a master set of room images or room characteristics

Once the imaging is complete, the cameras return to their stowed position. This position makes it visually clear to all room occupants that there is no recording possible or taking place. In some further variations, an independent actuatable camera housing that can open and close remotely may be provided to ensure that the cameras are blocked from capturing images during patient visits.

The cabinets and drawers that contain purchased product are inventoried by the system 1118, via local mechanical sensors, similar to those used in the hospitality industry by MINIBAR SYSTEMS (Rockville, MD) and AXXESS INDUSTRIES (Kelowna, B.C., Canada). This inventorying process recurs at the end of the clinic. At that time, the system may be configured to tally the difference in product-levels and compares this difference with any products purchased as part of the lease. If the numbers match, the system does not assess any extra charges; if there is a discrepancy, the physician's account will be charged the extra cost(s) of used products 1120.

### Extended Provisioning

If the physician has rented a locker, a refrigerator, a centrifuge or an autoclave they could also proceed to the storage/lab room. As this is a shared room, access to enter is disabled at all times. However, if the system detects the physician outside this door and, upon validating that their lease gives them the right to enter, proceeds to open the door. At the same time, the system may use the cameras inside the storage/lab room to take pictures of the condition of the room: snapshots of the floors, walls, countertops, lockers, autoclaves, centrifuges, etc. are taken. After completing this imaging, the system uses the cameras to continue to record video or monitor the storage room.

Once the physician is inside, the system increases the occupancy of the storage/lab room and proceeds to unlock any specific products leased by the physician: any locker(s) or refrigerator(s) are unlocked and any autoclave(s) or centrifuge(s) are enabled. They remain unlocked for the duration the physician is inside the room. Once the physician exits the room, the system locks the physician's rented locker(s) and/or refrigerator(s). Any autoclave(s) or centrifuge(s) rented will remain enabled for the duration of the physician's clinic. The system then takes pictures of the room's condition, ends videotaping of the storage room, and decreases the room's occupancy number.

If the physician has rented a nurse's station they or their staff can proceed to the nurse's room. As this is a shared room, access to enter is disabled at all times. However, if the system detects the physician or their staff outside this door via RFID or by camera, upon validating that their lease, the system can give the physician the right to enter and proceeds to open the door. At the same time, the system may use the cameras inside the nurse's room to take pictures of the condition of the room: snapshots of the floors, walls, furniture, printer, etc. are taken. After completing this imaging, the system uses the cameras to videotape the nurse's room. The system also increases the occupancy number of this room.

### On-Time Patient Processing

In some examples, when a patient arrives at the co-working site at their appointment time, the system matches the patient's arrival time with their appointment time slot and determines that the patient is on-time for their appointment. The system checks that their physician (or their physician's staff) is on site. If they are, then the system records the patient's arrival time. The kiosk dispenses an RFID ticket printed with the patient's name and appointment information, along with their physician's name and contact phone number.

If the corresponding physician (or their staff) is not on site, the kiosk tablet informs the patient of this and denies them entry. The kiosk tablet instructs them to wait outside of the facility until their physician checks-in and enters the site. The patient can only enter once their physician (or their staff) is on the premises. The display directs the patient to proceed to their assigned room using the door located in front of them and to the right.

The patient proceeds to enter their room. At this point, the system checks the location of the physician. If this is different from that of their patient, the system then alerts the physician that their patient has been seated in the specific room. The system updates the corridor tablet corresponding to the room with a status of occupied and an optionally a red-colored background. The system increases the occupancy number of the room, records the time the patient has entered the room and begins to track the duration the patient is in the room. If the physician is in the room, the system starts to track the duration the two are together. The system also updates the physician's status to Busy.

Once the appointment is finished, the patient leaves the room. The system decreases the occupancy of the room and updates the corridor tablet corresponding to the room with a status of unoccupied and a green-colored background. The system calculates the duration the appointment took. The physician's status is updated to Available. The patient is directed to the exit and exits the site. The system determines that the patient has exited, disables the patient's RFID ticket and decreases the number of patients the specific physician has on site. The system then reconfirms the exit with the dedicated People Counter sensor at the site's exit door. The system reduces the number of site occupants accordingly.

The system checks the physician's schedule to determine if there is a next patient waiting. If there is one, in one of the physician's leased rooms, the system notifies the physician of the next room they need to go to. If there is one other patient but in the waiting room, the system notifies the patient to come to the physician's current room.

### Late Patient Processing

In some examples, when a patient arrives at the co-working site, the system may match the patient's arrival time with their appointment time slot and determine that the patient is late for their appointment. The system determines that the patient's arrival time is still within the window of time which permits them to enter the facility. If the physician has an unoccupied rented space or room, the system directs the patient to this room using either the door on the left (leading to the waiting room) or the door on the right (leading to an exam/procedure room).

If the physician does not have any unoccupied rented space or room, the system informs the patient that they need contact their physician to reschedule their appointment. They are asked to do so off-site and informed that they cannot wait in the foyer.

The patient proceeds to enter the waiting room. The system begins to track the time the patient is in the room. The displays in the waiting room are updated to display the patient's identifier, their appointment time, their arrival time (in the waiting room), their physician's name and the physician's Busy status. The system also updates the occupancy of the waiting room and also alerts the physician that this patient is in the waiting room.

Once the physician's status becomes Available, the displays in the waiting room update to display their physician's Available status. The displays inform the patient of the room to which they need to proceed and directs them there through the doors leading from the waiting room to the appointment rooms. The system unlocks these doors and after the patient passes through either of them proceeds to lock them behind the patient. The system also decreases the occupancy of the waiting room.

### Early Patient Processing

In another example, a patient arrives at the co-working site. The system matches the patient's arrival time with their appointment time slot and determines that the patient is early for their appointment. The system determines that the patient's arrival time is still within the window of time which permits them to enter the facility. If the physician has an unoccupied rented space or room, the system directs the patient to this room using either the door on the left (leading to the waiting room) or the door on the right (leading to an exam/procedure room).

If the physician does not have any unoccupied rented space or room, the system informs the patient that they need exit the facility and return at their scheduled time. They are informed that they cannot wait in the foyer. The system begins to track the time the patient has first arrived on site and also alerts the physician that this patient has arrived and is waiting off-site.

Once they exit the facility, the system updates the occupancy of the foyer.

Once the physician's status becomes Available, the system contacts the patient and informs them of the physician's status.

### Early Multi-Patient Processing Using Waiting Room

In another example, the system begins normal processing, the same as though the patient was the first patient. The system compares the patient's arrival time with their appointment time slot and determines that the patient is early for their appointment but within the window of time which permits them to enter the facility. The system determines that according to the physician's lease the physician only has available room in the waiting room. The system may also determine the physician's status is Busy (seeing another patient). The kiosk tablet welcomes the patient and directs them to proceed to the waiting room using the door located in front of them and to the left.

The patient enters the waiting room. The system begins to track the time the patient is in the room. The displays in the waiting room are updated to display the patient's identifier, their appointment time, their arrival time (in the waiting room), their physician's name and the physician's Busy status. The system also updates the occupancy of the waiting room. The system alerts the physician of the patient's arrival time and current location.

Once the physician's status becomes Available, the displays in the waiting room update to display the patient's physician's status as Available. The displays inform the patient of the room to which they need to proceed and directs them there through the doors leading from the waiting room to the appointment rooms. The system unlocks these doors and after the patient passes through either of them proceeds to lock them behind the patient. The system also decreases the occupancy of the waiting room.

### Late Multi-Patient Processing Using Waiting Room

In other examples, when the system determines that the patient is late for their appointment, but within the window of time which permits them to enter the facility, the system determines that according to the physician's lease the physician only has available room in the waiting room. The system also determines the physician's status is Busy (seeing another patient). The tablet in the kiosk welcomes the patient and directs them to proceed to the waiting room using the door located in front of them and to the left. The system unlocks this door and after the patient passes through it proceeds to lock it behind them.

The patient enters the waiting room. The system begins to track the time the patient is in the room. The displays in the waiting room are updated to display the patient's identifier, their appointment time, their arrival time (in the waiting room), their physician's name and the physician's Busy status. The system also updates the occupancy of the waiting room.

Once the physician's status becomes Available, the displays in the waiting room update to display the patient's physician's status as Available. The displays inform the patient of the room to which they need to proceed and directs them there through the doors leading from the waiting room to the appointment rooms. The system unlocks these doors and after the patient passes through either of them proceeds to lock them behind the patient. The system also decreases the occupancy of the waiting room.

### Multi-Patient Processing Using Multiple Rented Rooms

In another example, the system begins normal processing, the same as though the patient was the first, but when comparing the patient's arrival time with their appointment time slot, the system determines that the patient is early for their appointment but within the window of time which permits them to enter the facility. The system determines that according to the physician's lease the physician has available room in both an exam room as well as the waiting room. The system also determines the physician's status is Busy (seeing another patient). The kiosk tablet welcomes the patient and directs them to proceed to the exam room using the door located in front of them and to the right. The system may be configured to prioritize the usage of the physician's exam/procedure/consolation rooms over the seats in the waiting room.

The patient enters the exam room. The system begins to track the time the patient is in the room. The tablet outside the room is changed to a red color background. The system also updates the occupancy of the room. The system alerts the physician of the patient's arrival time and current location.

Multi-Patient Processing when Clinician is Behind Schedule

A common clinic scenario involves multiple patients in queue when the physician is behind their clinic schedule. In some example, the system updates all of the physician's patient(s) in the waiting room, any exam or procedure room(s) and/or any patients in the parking lot of the physician's current status. The system calculates and informs them of estimated delay, which it calculates as the average time the physician has been spending up to that point with each patient. The calculation may be based on the physician's average prior patient appointment times computed by the system, or a selected subset of those if the physicians have different types of clinics or procedure appointments, or based on different days of the week. The system repeats this process every configured number of minutes (the default is the number of minutes the specific physician has allocated for their appointment time).

Multi-Patient Processing when Clinician is Ahead of Schedule

In a potential scenario where the clinician may be potentially ahead of schedule due to last-minute cancellations or unfilled appointment slots, the system may update all of the physician's patient(s) in the waiting room, any exam or procedure room(s) and/or any patients in the parking lot of the physician's current status. If the physician has another patient scheduled next, the system will have already directed that patient to wait in the waiting room (each physician receives one complimentary seat in the waiting room); the system updates the display in the waiting room with instructions for that patient to proceed to the free room. At the same time, the system may instructs patient(s) waiting outside the building to proceed to the site. The system determines the number of patients to be directed such based on the physician's number of leased rooms (including a physician's office—which depending on the physician's specification could be used to seat waiting patients), the number of corresponding seats in the waiting room, and the availability of each of these locations. The system informs all other patients of the estimated speed-up, which it calculates as the average time the physician has been spending up to that point with each patient. The system repeats this process every configured number of minutes (the default is the number of minutes the specific physician has allocated for their appointment time).

### End-of-Rental Period Processing

At the end of the lease period, each physician is given a free period of time (system configured to be 15 minutes) to gather their belongings and leave the premises. Depending on their lease terms this time might be spent on cleaning up any room(s) leased, putting away any rented sharps disposal container(s), packing any supplies they have brought along, storing their belongings in a rented movable cart in the room, storing their belongings in a rented locker, storing perishable supplies in a rented refrigerator, picking up sterilized utensils from any rented autoclaves, picking up patient specimen jars from the lab, picking up test tubes from any rented centrifuge(s), and/or placing any specimen(s) in lab pickup boxes (if they have signed up for this service).

As noted previously, at the end of this period, the system performs a visual inspection and inventory of each room. The video cameras take pictures of the configured locations in each space. The system compares these pictures with their counterparts taken before the beginning of the lease. Based on the difference(s) in these images, the system may determine the cleanliness status of the room and therefore any extra charges the physician must be assessed (his step is only performed only for leases which have not opted for the “Cleaning service” option).

Next, the system inventories all the products in the drawers and/or on shelves: each of these locations has its dedicated RFID antenna and each product in each location has its own RFID tag. At the end of the lease the system determines used inventory as follows: if an item's current location is different from the item's previous location, then the system counts the item as used by the physician. The logic being that a physician's removal of an inventory item from its location (even if momentarily) is captured by the system as a change in location. The RFID antenna at the given location will record the removal since it will no longer sense the item; even if the item is returned to its location the two locations will differ from each other (current being in the drawer and last being unknown). This precludes physicians from using product and replacing empty packaging.

Similarly, the system may determine whether the sharps disposal container was used—the system uses the RFID tag affixed to the container to determine if it was relocated from its stowed position (if the current and last positions differ).

Next, if the physician has any rented movable carts they are locked so their contents are safe from any access. If the specific room is rented to another physician and those have their own rented movable carts, the system alerts the facility manager that the current cart needs to be moved (and indicates the location) and that the next physician's cart must be relocated to the current room (and indicates that cart's current location).

Depending on the inventory levels in the vacated room the system also informs the facility manager of the items it needs to restock in the room.

### Mobile Storage Cart

In some variations, the lease customization may also include the option for locked mobile cart. A mobile cart would allow the user to maintain onsite storage of their own items and products onsite between their scheduled clinics, and the ability to easily move the cart to their assigned room(s) during scheduled clinics. In FIG. 1, for example, the carts 146 may be kept in a user accessible storage area 170 or room. The clinician or staff member can roll the cart 146 from the storage area 170 to the assigned room for the scheduled clinic. In FIGS. 3A and 3B, the cart 146 may be pushed into the room 300 and located against the side of the fixed lower cabinet storage 308.

In further variations, the cart 146 may be sized so that the top of the cart 146 is aligned with the countertop of the cabinet 308 in the room 300, and the front of the cart 146 is aligned with the front of the cabinet 308, when the cart 146 is stowed in the room 300. The cart 146 includes a plurality of drawers 310 or cabinets/shelves, with a plurality of caster wheels 312. In some variations, each drawer 310 may be individually lockable locally (e.g. by key or fob) and/or remotely (e.g. electronically via app and/or web interface), or may be globally lockable locally or remotely, with all of the drawers or cabinets being locked or unlocked together. To facilitate monitoring and/or control of the cart 146, wireless or wired sensors, actuators, and connectors may be provided in the cart, storage area 170 (FIG. 1) and/or examination room (FIGS. 3A and 3B). In some further embodiments, the cart may be optionally provided with power via a battery or power cords to charge equipment during storage or to provide power during clinic use.

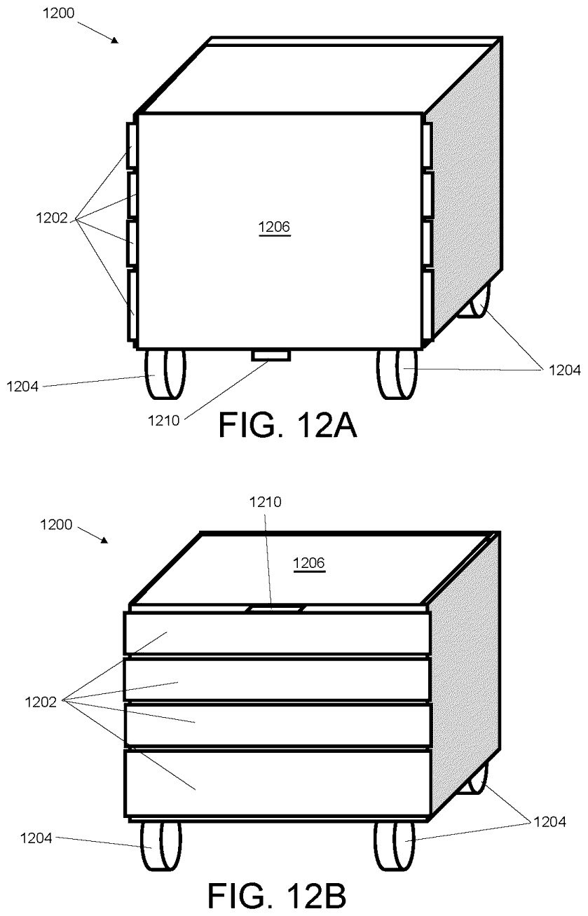

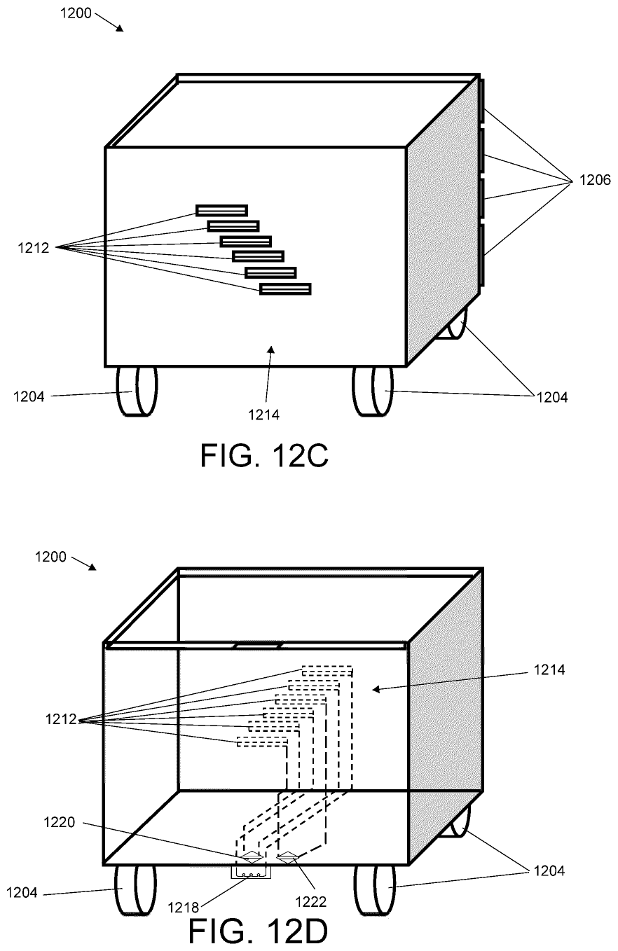

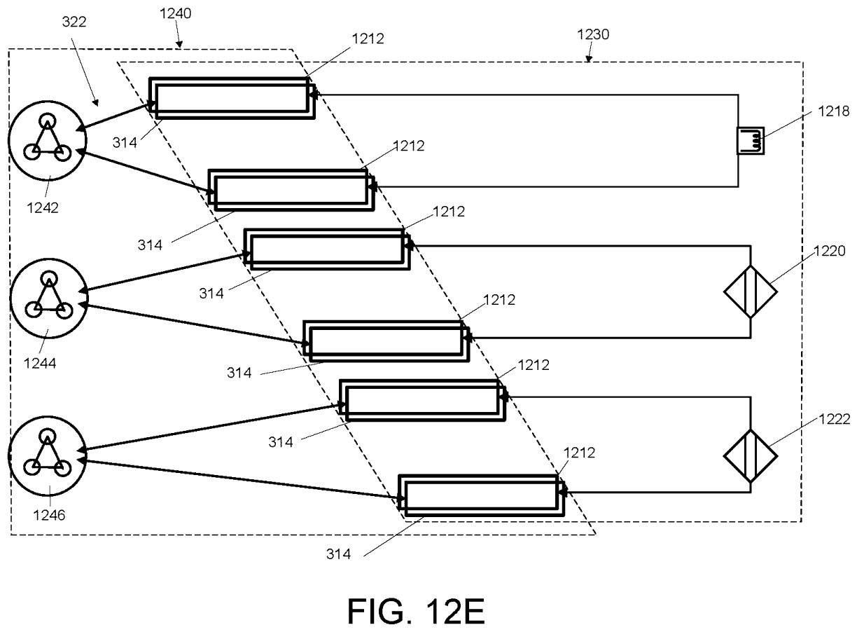

In a further example of a mobile cart configuration, depicted in FIGS. 12A to 12D, the cart 1200 comprises a plurality of pull-out drawers 1202 and wheels 1204, as well as a security door 1206 that can cover the drawers 1202 and reversibly and remotely secured via an electronically actuatable lock 1208. The security door 1206 may be a flipper-style door that comprises a closed configuration depicted in FIG. 12A, with a closed configuration where the door 1206 is covering the face of the drawers 1202, and an open configuration where the door 1206 is flipped up from the upper edge and slid back onto the top of the cart 1200. In other variations, the door may be two doors and configured to swing open flush to the sides of the cart, or may have a multi-segmented roll top configuration. The door 1206 may comprise a handle 1210 to facilitate it opening and closing. The handle 1210 may be located on or near the front edge of the door 1206.

The cart 1200 may include a passive or active RFID or other identification tag which is detectable by any proximity sensors located through the co-working facility, so that the location of the cart 1200 may be tracked by the system. In some variations, the RFID tag may be an ultra-high frequency (UHF) system. In other variations, the geolocation system may be a KNX home automation, WiFi, Bluetooth or NFC-based system. In some further embodiments, the cart 1200 may have wired connectors or contacts to further facilitate access to sensors located in the cart 1200. In FIG. 12C, for example, the cart 1200 includes a plurality of surface contact connectors 1212 located on a rear surface 1214 of the cart, The exemplary surface contact connectors 1212 in FIG. 12C are located on offset or Z-shaped elongate clip strips which may be used to engage complementary surface contact connectors 314 located on offset or Z-shaped elongate clip strips attached to a wall where the cart 1200 is to be positioned.

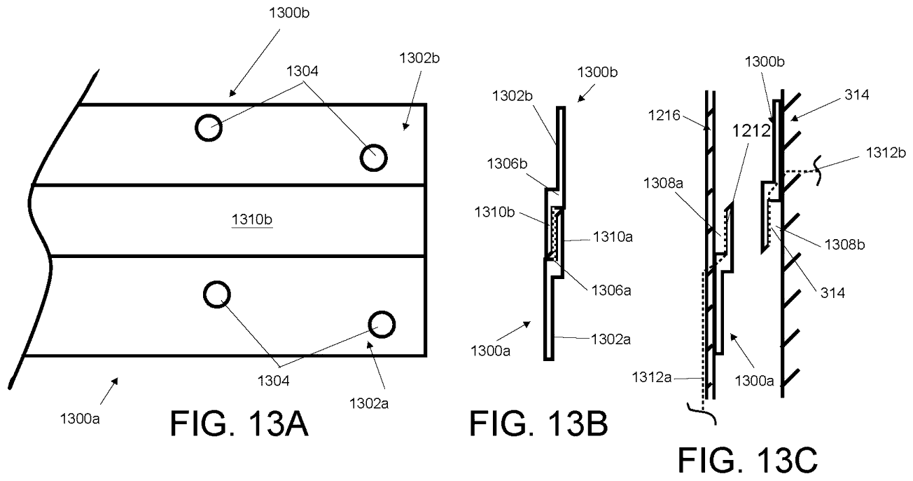

FIGS. 13A to 13C depict the structure of the complementary clip strips 1300a, 1300b and surface contact connectors 1212 of the cart and of the surface contact connectors 314 of the wall. Although the specific example depicted in FIGS. 13A to 13C is configured with a lower clip strip 1300a for the wall 1216 of the cart, and an upper clip strip 1300b for the room wall 318, on other variations, the relationship may be reversed. Each of the clip strips 1300a, 1300b including a mounting section 1302a, 1302b with one or more fastener openings 1304 to attach the clip strip 1300a, 1300b to the desired structure, e.g. cart wall 1216 or room wall 318. An offset section 1306a, 1306b extends outward to form a cavity 1308a, 1308b, in which the complementary engagement sections 1310a, 1310b of the other clip strip 1300b, 1300a may be received. To engage the cart to the wall, the rear surface of the cart 1214 is brought against the room wall 318 and is slid parallel to the wall until the complementary clip strips 1300a, 1300b begin to overlap and engage. A stop surface from adjacent fixed furniture or an alignment structure attached to the wall may be provided to stop further translation of the cart when the desired overlap, the surface contact connectors 1212 and 314 that are located on the engagement sections 1310a, 1310b are then in electrical continuity to pass power and/or data signals. The height of the engagement sections 1310a, 1310b relative to the height of the cavities 1308a, 1308b may be smaller, and the depth of the cavities 1308a, 1308b may be greater than the thickness of the of the clip strip 1300a, 1300b, to provide some height and depth tolerance into the alignment of the cart and the wall, so that the engagement of the cart does not require precise positioning and alignment. In some variations, the total height of the each clip strip 1300a, 1300b may be 2 to 4 inches, and the length may be 6 to 8 inches. Although the large surface area of the electrical contact connectors 1212, 314 permit a significant degree of mounting variability while still maintaining the electrical connection. To further ensure electrical continuity between the surface contact connectors 1212 and 314, a structure on the room wall may be provided to slightly angle the cart away from the wall after initial or partial engagement between the clip strips 1300a, 1300b is achieved, to facilitate contact and engagement of the surface contact connectors 1212 and 314. In FIGS. 3A and 3E, for example, as the cart is slid into engagement, the cart will eventually reach the wall wire faceplate 320. The wall wire faceplate 320 may include an outwardly angled ramp surface to slightly angle to cart away from the wall, thereby increasing contact between the surface contact connectors 1212 and 314 against each other. The increasing resistance from the ramp surface may also help to resist rollback of the cart out of alignment and engagement with the wall connectors.

The wiring 1312a, 1312b of the surface contact connectors 1212, 314 may be embedded in the attached wall 1216, 318 of the cart or wall, respectively, as depicted in FIG. 13C. Alternatively, as depicted in FIGS. 3C and 3E, the wiring 322 or may be partially surface mounted between a shared wall opening at the faceplate 320, so that only a single wall opening is required for the plurality of wires 322.

Referring to the exemplary examination rooms 118 in FIG. 1, the intended cart locations relative to the door of the examination room and fixed cabinets may require a cart 146 that can be engaged to the wall by sliding from either the left or the right when facing the cart location. For example, in FIG. 3C, a cart would have to be rolled from right to left in order to engage the surface connectors 314, while in FIG. 3D, a cart would have to be rolled from left to right. While the arrangement of the surface connectors 314 is the same for each room type, the relative location of the faceplate 320 will be different and located farthest from the starting cart location and closer to any stop structure. For example, in FIG. 3C, where the cart is moved from right to left, the faceplate 320 is located to the left of the connectors 314, while in FIG. 3D, where the cart is moved from left to right, the faceplate 320 is located to the right of the connectors 314.

To facilitate the engagement, in some variations the surface connectors 1212, 314 may be configured in a staggered arrangement as shown in FIGS. 3C, 3E and 12C to 12E. In the particular arrangements shown, each subsequent connector 1212, 314 is offset in the same direction to thereby form a slanted connector arrangement, but in other variations, the staggered arrangement may be an alternating arrangement, or a vertically aligned arrangement may be provided. It is hypothesized that the staggered arrangement, and in particular the slanted staggered arrangement by reduce the risk that any surface mounted wiring from the connectors 1212, 314 may be sheared or damaged by the clip strips 1300a, 1300b.

Referring to FIG. 12D, the exemplary cart 1200 may be configured with an electronically actuatable door lock 1218, which may be actuated wirelessly, or as shown in FIGS. 12D and 12E, through the surface contact connectors 1212 of the cart 1200. The cart 1200 is also configured with a cart door lock sensor 1220 to detect whether the door lock 1218 is engaged or disengaged, and a cart door position sensor 1222, to determine whether the door is closed or open. The door lock and door position sensors 1220, 1222 may be mechanical switch sensors, electrical contact sensors, or Hall-effect sensors, for example. FIG. 12E is the electrical schematics of the cart electrical system 1230 and the wall electrical system 1240. The lock 1218 is in electrical continuity with two of the surface contact connectors 1212, which are configured to contact and bridge with two corresponding surface contact connectors 314 of the wall electrical system 1240. These connect in turn to the cart lock interface 1242 controlled by the provisioning module. Likewise, the dock lock sensor 1220 is also in electrical continuity with two of the surface contact connectors 1212, which are configured to contact and bridge with two corresponding surface contact connectors 314 of the wall electrical system 1240. These connect in turn to the cart lock sensor interface 1244 to provide lock status to the provisioning module. Finally, the cart door cart door position sensor 1222 is in electrical continuity with two of the surface contact connectors 1212, which are configured to contact and bridge with two corresponding surface contact connectors 314 of the wall electrical system 1240. These connect in turn to the cart door sensor interface 1246 to provide lock status to the provisioning module. Using the sensor information from the cart door position sensor 1222, the provisioning module is able to confirm cart door closure before attempting to lock the cart door, and it also able to confirm when the door lock 1218 is actuated, whether locking was successfully achieved via the door lock sensor 1220. If unsuccessful, the provisioning module and/or the orchestration server 500, describe below, may contact the user via in room display 302a/302b to reattempt cart door closure and/or locking before the cart is re-stowed after a scheduled clinic, for example.

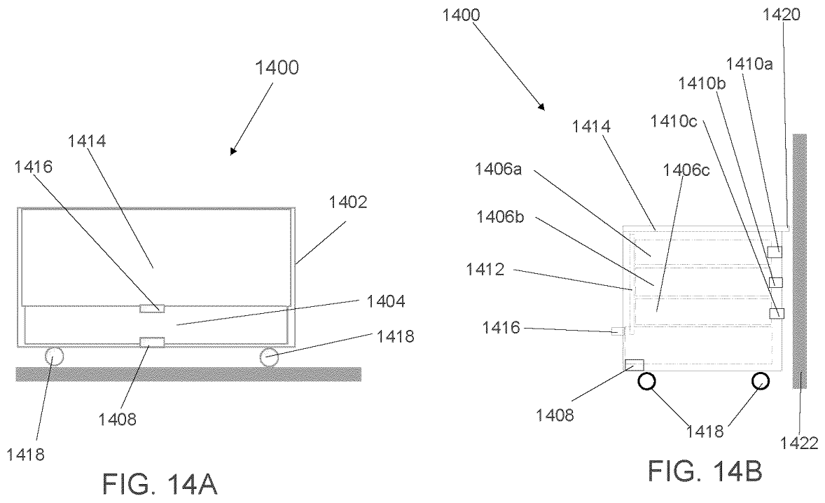

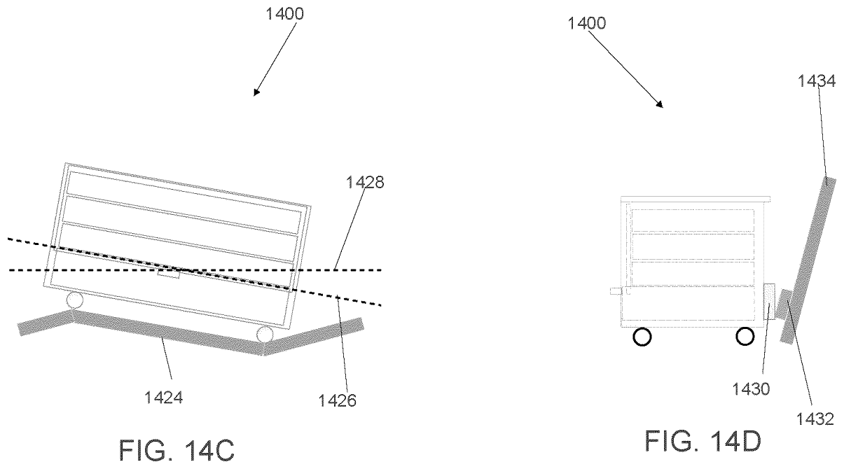

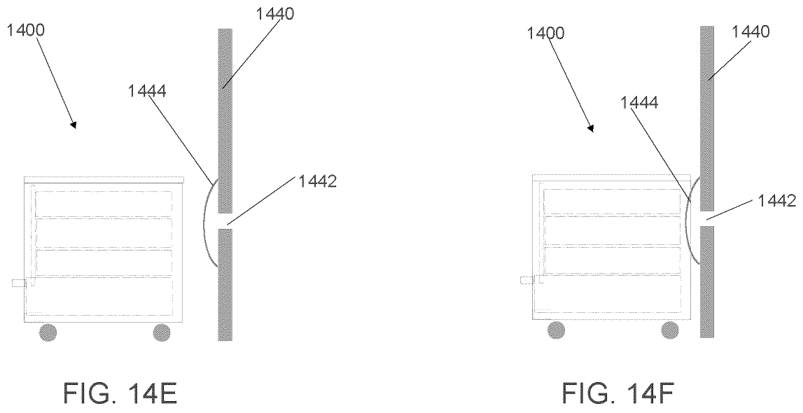

FIGS. 14A and 14B schematically depict another exemplary embodiment of a mobile cart 1400, comprising a main housing 1402, main drawer 1404, secondary drawers 1406a-c, main drawer remote lock 1408, secondary drawer remote locks 1410a-c, cart door 1412 with pull handle 1414, cart top 1416 and wheels 1418. In FIGS. 14A and 14B, the external contacts and optional rear anchor are not depicted, but any of the contact and anchor configurations described herein and below may be implemented with the cart 1400. In this embodiment, the cart door 1412 only covers the secondary drawers 1406a-c, leaving the main drawer 1404 exposed and permitting access to the main drawer 1404 independent of whether the door 1412 is open or closed. The main drawer 1404 still includes a lock 1408, while the secondary drawers 1406a-c each include their own drawer lock 1410a-c that may be independently lockable/unlockable. This permits optional equipment and items to be accessible to the user that can be immediately accessible in the room to be provided via the cart, instead of in a lockable compartment within the room itself. The cart door 1412 may also optionally comprise a remote lock (not shown). The cart door 1412, when opened is configured to reside between the cart top 1414 and the topmost drawer 1406a. The cart top 1416, is provided with a rear overhang 1420. This overhang 1420 may limit contact between the wall 1422 and the rear surface of the main housing 1402, which may facilitate anchoring and/or protect any rear external contacts from inadvertent damage during use.

FIGS. 14C and 14D also depict other use scenarios that may result in suboptimal connection between the cart and control system. In some buildings, the floors and walls may not be consistently perpendicular to each other and/or level. Variations may occur due to building structural issues with the concrete or steel beams, for example, or post tension slab construction may result in floor sagging between structural walls and posts. In the exaggerated uneven floor 1424 depicted in FIG. 14C, The connectors along the back of the cart 1400 would be parallel to the cart orientation and therefor have a tilted angle 1426 from the horizontal angle 1428, while the connectors long the wall may be set to the horizontal angle 1428, thereby resulting in a mismatch between the angles 1426 and 1428. This mismatch may result in damage to the connectors and/or interruption in signaling. FIG. 14D schematically depicts another construction variation that may be found between rooms, depicting in an exaggerated illustrative fashion misalignment of the cart connectors 1430 and the wall connectors 1432 when the wall 1434 is not perpendicular, and illustrating why the connectors and/or attachment components of the cart and/or walls may be configured to accommodate a range of variability.

FIGS. 14E and 14F schematically illustrate another potential construction variability that may affect connectivity of the cart 1400. In some jurisdictions, such as the United States, commercial and/or medical wall construction may involve drywall board 1440 screwed to wood or metal wall studs. The drywall boards have edges that may be uneven or inconsistent in thickness that may be addressed during constructions by providing a gap or seam 1442 that may be filled will drywall compound and covered with drywall tape 1444. This may result in horizontal and/or vertical bulges in various locations of a wall for which the attachment components may be configured to accommodate.

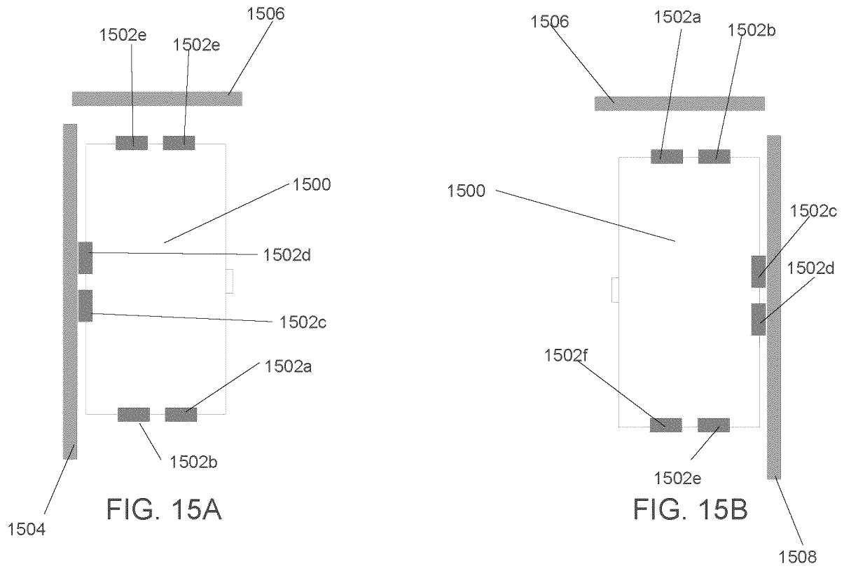

Even under ideal construction consistency, cart connectivity may still affected by room configuration, e.g. the “handedness” of the intended docking location of the cart and how the cart may be limited in the approach to the docking location based on adjacent walls, furniture or equipment. Carts have drawers that are accessible on one side of its housing, which will necessarily be the opposite from the wall to which the cart is to be connected. However, the location may not always be accessible from both the left and right of the location. In the cart 1500 schematically depicted in FIG. 15A, for example, side connectors 1502a-b located on the left side of the cart when docked at the intersection of a left-sided wall 1504 and front wall 1506 (or furniture blocking access from that side). The same cart 1500 connected to a location with a right-sided wall 1508 intersecting with the front wall 1506, the left side connectors 1502-ab are now facing the front wall 1506 rather than the use. Even though the rear connectors 1502c-d may be connected to either the left wall 1504 or right wall 1508, it will be noted that potential issues may still exist because the low-voltage systems that may be used for the connectivity functionality is often a Direct Current system, and the powered components may not intrinsically work when the positive and negative power connections are reversed, as they may be with different orientation walls 1504, 1506.

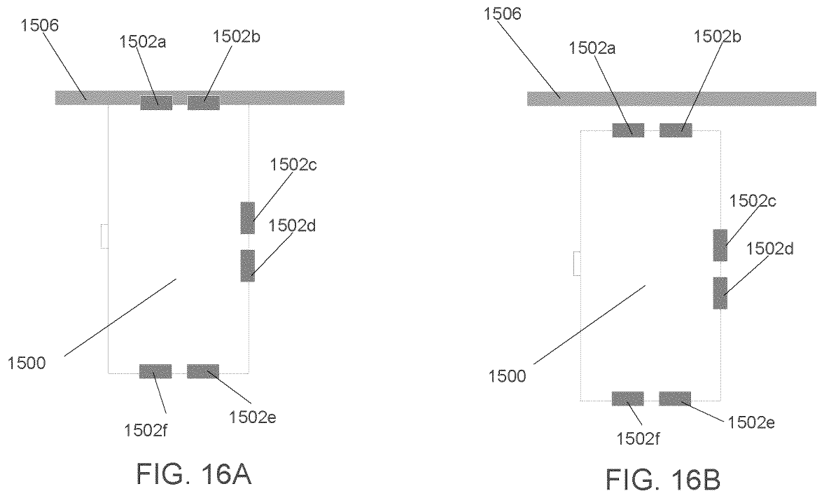

Cart connectivity may also be subject to signal loss or other issues as a result of adjacent activities. For example, during use, the user other personnel may inadvertently bump cart. Vibrations from the use of elevators, stairs or other nearby activities may result in minor cart displacement. The opening and closing of the drawers may also cause some cart movement. Furthermore, repeated cycles of attachment and detachment of the carts to the wall over time may results in deformation or other damage to the anchors and/or external contacts of the cart. Any of these alone or together may result in suboptimal or interruption of the control and status signals between the cart and the control servers, or even damage to the connection hardware. FIGS. 16A and 16B, for example, depict engagement and disengagement of side connectors 1502-ab of cart 1500 that may result from bumping or drawer opening/closing. This could result in an inconsistent user experience with the cart door and/or drawers opening/closing or failure to provide or block access. Without accurate connectivity, the control system may not accurately receive signals regarding the state of the cart remote locks or other sensors.

To address the various potential connectivity issues, one or more z-clips on the cart and/or wall may be mounted on a spring, to bias the z-clip to an extended or retracted position but to resilient allow the z-clip to retract or extend, respectively. The spring may be a leaf spring, accordion spring or a coil spring, for example. As noted elsewhere herein, the z-clips may be provided with minimum heights to the upper and lower portions of the z-clip to accommodate different vertical engagement heights between the cart and wall.

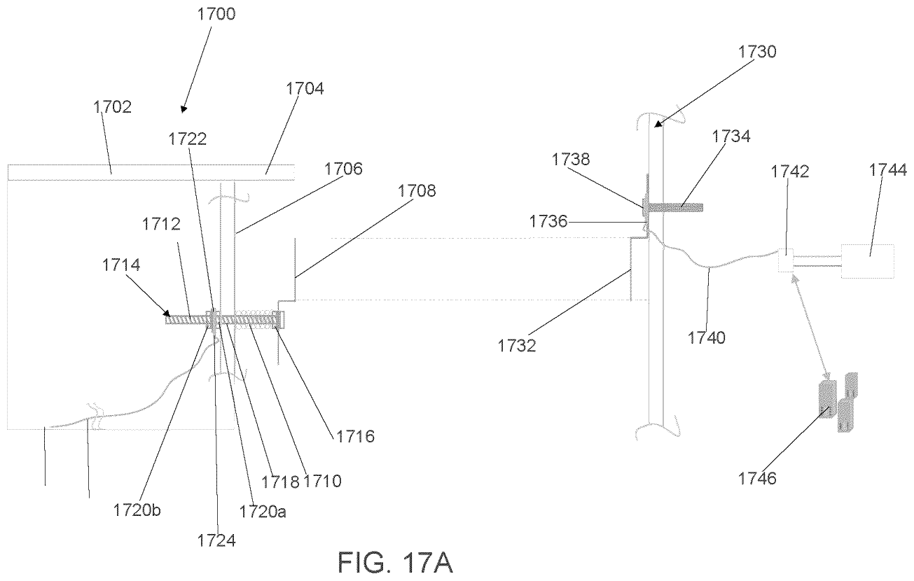

FIG. 17A is a schematic close-up view of a spring-based cart clip and complementary wall clip. The cart 1700 comprises a cart top 1702 with an overhang 1704 from the rear cart wall 1706. The distance between the overhang 1704 and the rear cart wall 1706 may be equal to or greater than the cart z-clip 1708 in the extended position. The spring 1710 is located between the rear cart wall 1706 and the Z-clip 1708 with the shaft 1712 of the screw 1714 extending through them. The clip 1708 is fixedly attached to the screw 1714 by an external fixation nut 1716, but the screw shaft 1712 is able to translate in and out of the wall opening 1718 in the rear cart wall 1706. Although the shaft 1712 depicted in FIG. 17A is fully threaded along its length, in other variations, an unthreaded middle portion may be provided, to facilitate its translation in the wall opening. The extension distance of the screw 1714 is set by the location of two internal nuts 1720a-b located between the distal end 1722 of the screw 1714 and the rear cart wall 1706. Between the nuts 1720a-b are an optional washer 1722 and also the electrical eyelet or ring connector 1724, which then connects via a wire or cable 1726 to a lock 1728, sensor or battery of the cart 1700.

On the corresponding wall 1730, a complementary z-clip 1732 is attached to the wall 1730 via a clip screw 1734. A corresponding eyelet connector 1736 is provided between the z-clip 1732 and the screw head 1738, and connected via a wire 1740 to a control relay 1742. In other variations, the electrical eyelet connector 1736 may be provided on other side of the wall 1730 and attached to the screw 1734 via one or two nuts. The relay 1740 may be connected to an AC or DC power supply 1744 and may communicate with the main controller 1746 or an intermediate relay controller via a wired or wireless connection. The z-clips 1708, 1732 may comprise a minimum vertical height of 1″, 1.5″ or 2″, or be in the range of 0.75″ to 5″, 1″ to 3″, or 1″ to 2″. The horizontal offset distance of the z-clip 1708, 1732 may be 0.25″, 0.5″, 0.75″ or 1″, or be in the range of 0.25″ to 1″, 0.25″ to 0.75″, or 0.25″ to 0.5″. The length of the z-clip may 1″, 1.5″, 2″, 3″, 4″ or 5″, or be in the range 1″ to 5″, 1″ to 4″, 1″ to 3″ or 2″ to 4″, or 2″ to 3″. The overhang distance and/or the cart Z-clip extension distance may be 0.5″, 0.75″, 1″, 1.25″ or 1.5″, or be in the range of 0.5″ to 1.5″, 0.5″ to 1″ or 1″ to 2″. FIG. 17B depicts a variation of the cart 1700 comprising two cart z-clips 1708 and 1750 coupled to the lock 1728, sensor or battery via wires 1752a-b, and configured with two corresponding wall Z-clips 1732, 1760 attached to the wall 1730 and both connected by wires 1762a-b to the relay 1740.

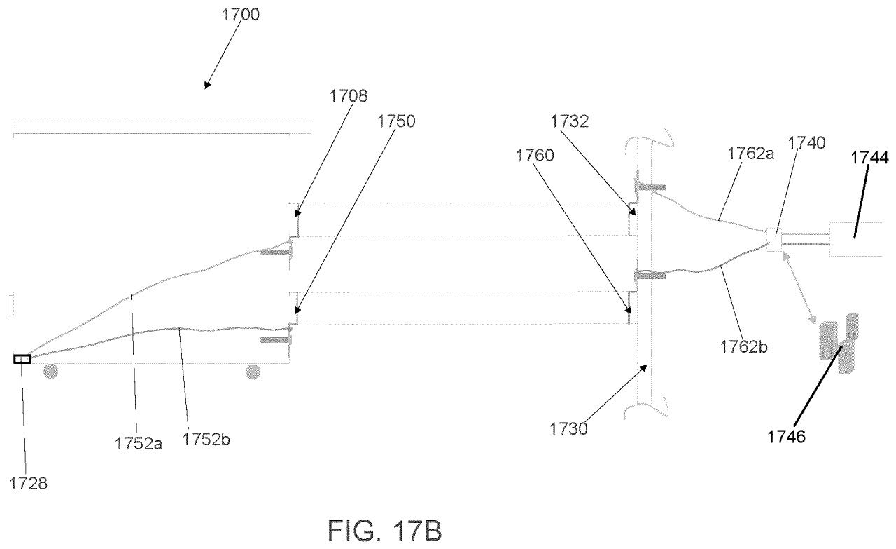

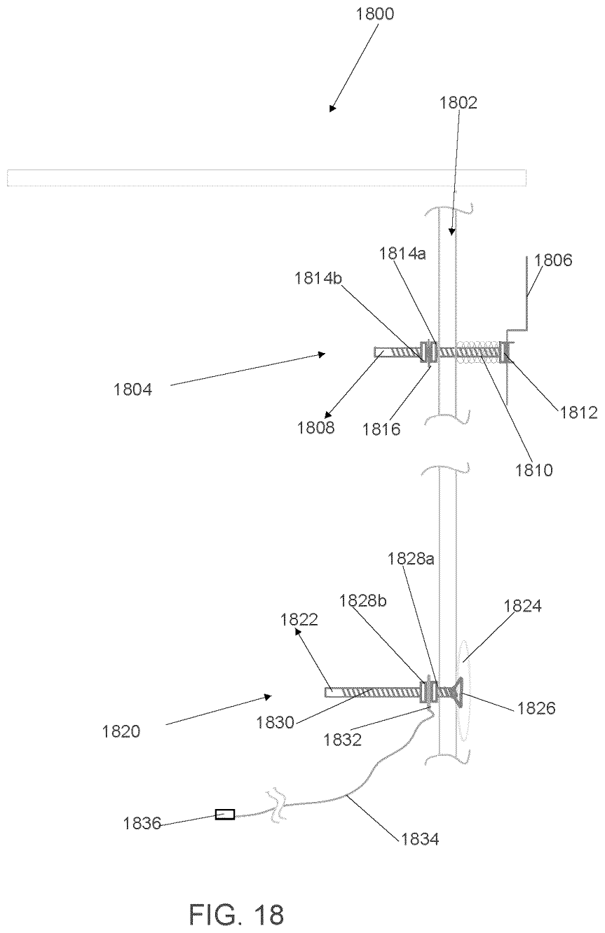

Although two sets of cart and wall z-clips are provided in the embodiment depicted in FIG. 17B, in other variations, only one set may be provided, or in still other variations, no sets of z-clips may be provided. In these other variations, non-locking, optionally spring-based electrical contact connectors may be provided. In FIG. 18, for example, the cart 1800, the rear cart wall 1802 may be provided with a first or superior z-clip assembly 1804, similar to the z-clip arrangement found in FIG. 17A, with corresponding z-clip 1806, screw 1808, spring 1810, external nut 1812, two internal nuts 1814a-b and electrical eyelet connector 1816. A second or inferior contact assembly 1820, however, comprises a retention screw 1822 with a circular metal contact member or plate 1824 located between the rear cart wall 1802 and the screw head 1826. In some variations the contact member 1824 may be planar or may be curved or frusto-conical in shape to provide resilient contact with a corresponding contact member attached to the wall, and may optionally have a square, star or polygonal shape. The screw 1822 may be retained on the rear cart wall 1802 by two retention nuts 1828a-b along the threaded shaft 1830 of the screw 1822, and an eyelet connector 1832 therebetween. The eyelet connector 1832 is connected via a wire or cable 1834 to a cart lock 1836, sensor or battery. In this embodiment, no spring is provided, but in other variations, the contact assembly 1820 may have a similar configuration as the z-clip spring assembly 1804, but with the contact member in place of a z-clip.

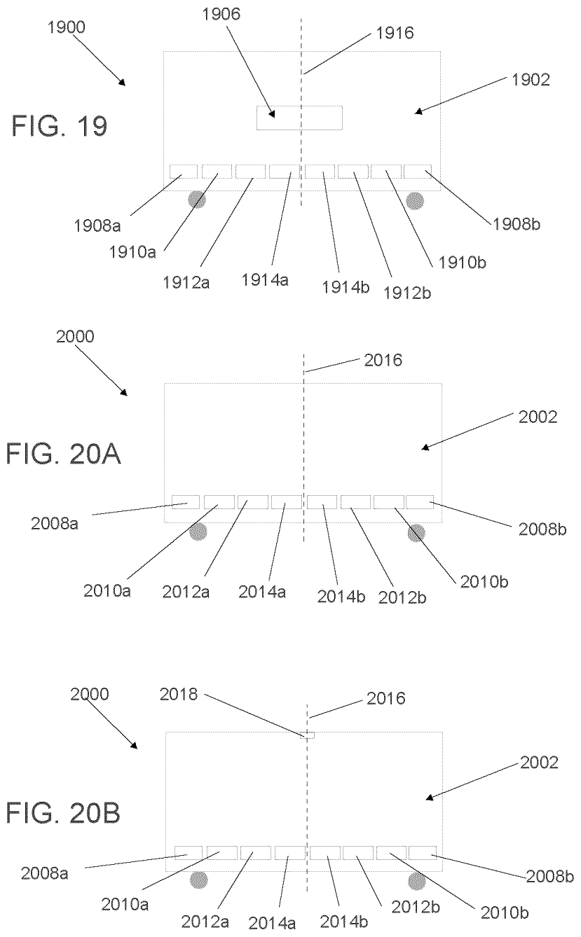

FIG. 19 schematically illustrates one exemplary embodiment of the connector arrangement on the rear wall 1902 of a cart 1900 that may be provided with any of the cart embodiments herein. In this embodiment, a z-clip anchor 1906 is provided in the center region of the rear wall 1902, but is not electrically connected and is used only for alignment and attachment of the cart 1900 to a docking wall location. Along a horizontal inferior region of the rear wall 1902, a set of voltage connectors 1908a-b are provided, along with one or more pairs of signal or electrical connectors 1910a-b, 1912a-b and 1914a-b. Because of the presence of the center z-clip anchor 1906, the signal connectors 1910a-b, 1912a-b and 1914a-b may comprise the contact member-type connector illustrated in FIG. 18, rather than the z-clip connector depicted in FIG. 17A, but in other variations, one or more of the signal connectors 1910a-b, 1912a-b and 1914a-b may be z-clip type. In this embodiment, the z-clip 1906 and the pairs of voltage and signal connectors 1908a-b, 1910a-b, 1912a-b, 1914a-b are symmetrically arranged relative to the midline 1916 of the cart 1900, so that the cart can be used with different “handedness” room configurations. In this embodiment, to avoid any polarity issues with using DC voltage source, the cart 1900 may be configured to accept AC voltage, so that the voltage connectors 1908a-b avoid any polarity issues that would be affected by use of a DC power source. In other variations, however, the wall voltage connectors may be reversed, based on the expected docking direction of the cart specific to a room configuration.

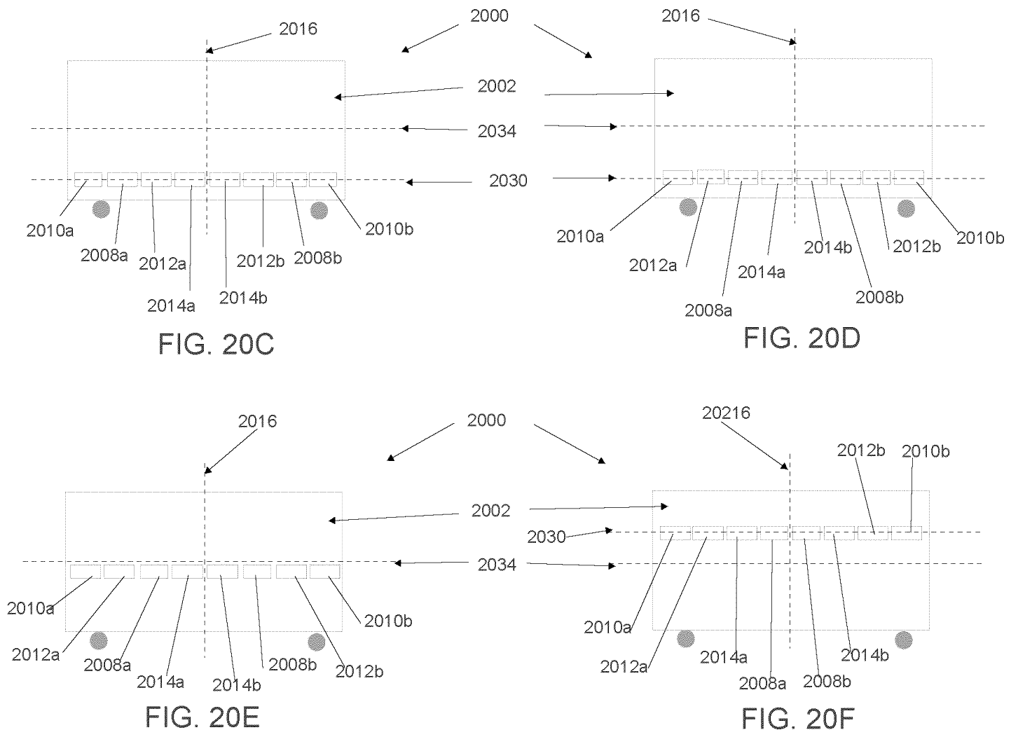

In FIG. 20A, the connector arrangement on rear wall 2002 of the cart 2000 lacks a center passive z-clip anchor compared to cart 1900, so the at least one pair of the voltage connectors 2008a-b and electrical connectors 20910a-b, 2012a-b and 2014a-b are provided as z-clip type connectors as in FIG. 17A. In some further examples, voltage connectors 2008a-b are z-clip type, and electrical connectors 2010a-b, 2012a-b and 2014a-b are optionally or are also all z-clip type, with any remaining connectors comprising contact member type.

In FIG. 20B, the connector arrangement on rear wall 2002 of the cart 2000 may be similar to that depicted in FIG. 20A, but wherein the rear wall 2002 further comprises an identifying tag 2010, such as a QR code, NFC tag, Bluetooth beacon, RFID tag and the like. The tag 2018 may be provided along the midline 2016 of the cart 2000, so as to provide a consistent tag location that is independent of the docking direction or orientation of the cart 2000 relative to the wall. In some variations, the tag 2010 may be provided along the top edge or section of the rear wall 2002, to reduce or avoid any electrical, RF or spatial interference with the voltage connectors 2008a-b and/or electrical connectors 2010a-b, 2012a-b and 2014a-b.

FIGS. 20C to 20F depict further variations of the connector arrangement for the mobile cart. In FIGS. 20C and 20D, for example, the voltage connectors are located at the inner symmetrical locations relative to the midline 2016, compared to the variation depicted in FIG. 20A. FIGS. 20E and 20F depict further variations where the connectors remain aligned along a horizontal line 2030 but wherein the horizontal line is located closer to the horizontal midline 2034 or the upper edge of the rear wall 2002 or between the upper edge and the horizontal midline 2034, respectively.

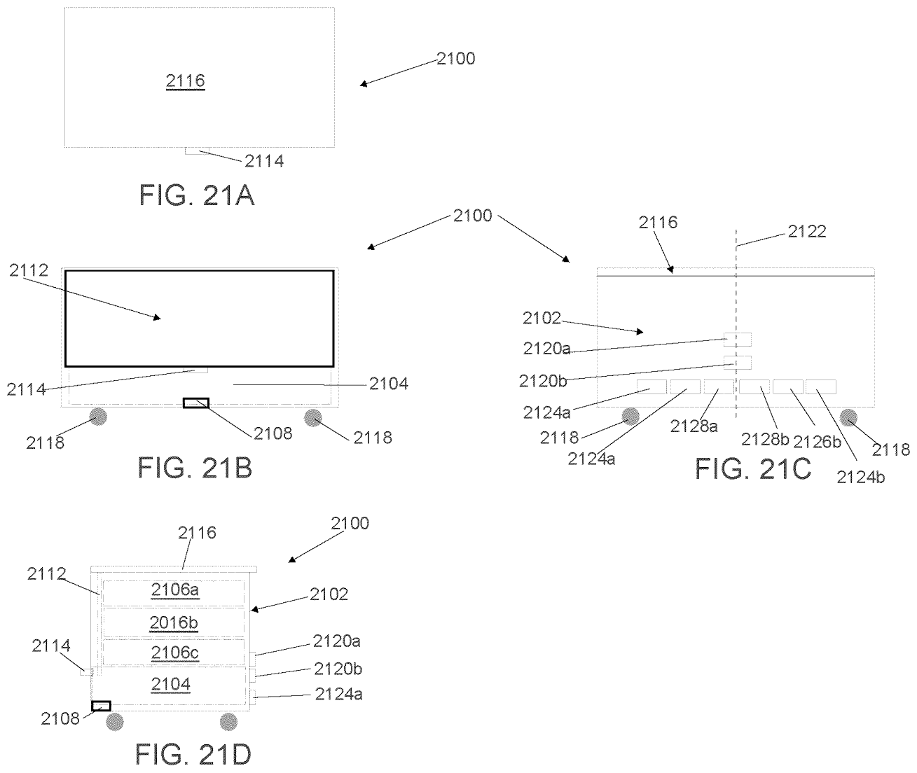

In FIG. 21A to 21D, the cart 2100 may be similar to other carts herein, comprising a main housing 2110, main drawer 2104, secondary drawers 2106a-c, main drawer remote lock 2108, optional secondary drawer remote locks (not shown), cart door 2112 with pull handle 2114, cart top 2116 and wheels 2118. The connector arrangement on the rear wall 2102 of the cart 2100 differs from those in FIGS. 19, 20A and 20B in that the voltage connectors 2120a-b are located along the midline 2122 of the cart 2100. The remaining electrical connectors 2124a-b, 2126a-b and 2128a-b are also symmetrically configured with respect to the midline 2122. In this configuration, the voltage connectors 2120a-b can be powered without regard to polarity of the connectors or docking orientation of the cart 2100. For example, the upper voltage connector 2120a may be the positive electrode while the lower voltage connector 210b may be the negative electrode, or vice versa. The connectors 2120a-b, 2124a-b, 2126a-b and 2128a-b may be z-clip type or contact member-type.

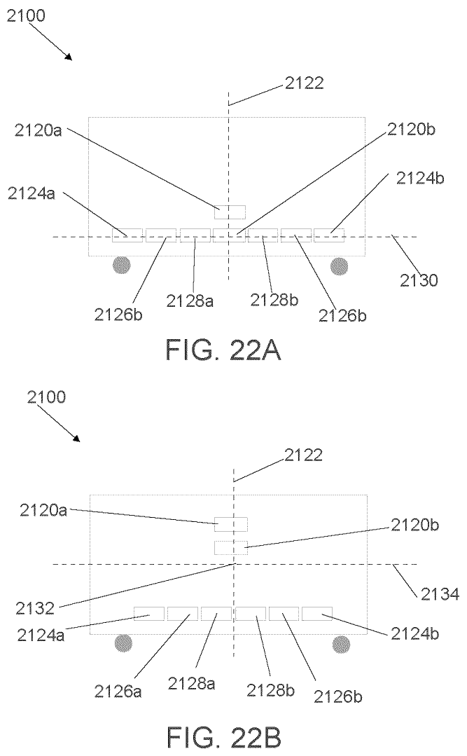

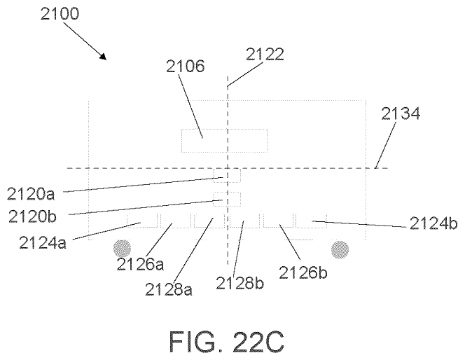

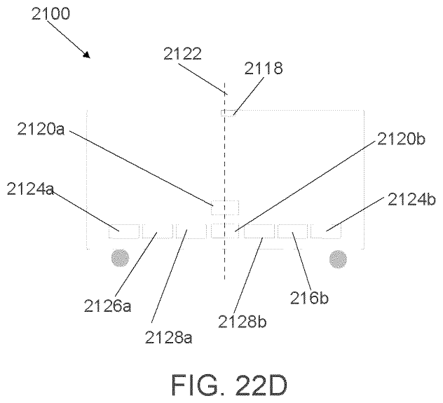

FIGS. 22A and 22B depict additional variations of a midline symmetric connector configuration. In FIG. 22A, while both voltage connectors 2120a-b are located along the midline 2122, the lower of the voltage connectors 2120b is also aligned along a horizontal line 2130 with the other connectors 2124a-b, 2126a-b and 2128a-b. The upper voltage connector 2120a may be the negative voltage connector, while the lower connector 2120b may be the positive voltage connector, but in other variations the polarity may be the opposite. FIG. 22D is similar to the cart 2100 depicted in FIG. 22A, but with the addition of an identification tag 2118 to the cart 2100. The tag 2118 is located at the top of the vertical midline of the 2122, but in other variations the tag may have a lower position on the rear wall or may be offset from the vertical midline. The In FIG. 22B, both voltage connectors 2120a-b are located along the midline 2122 and both are offset from the one or more pairs of other electrical connectors 2124a-b, 2126a-b and 2128a-b but are located in the upper region of the rear wall 2102, above the centerpoint 2132 of the vertical midline 2122 and horizontal midline 2134, compared to the configuration depicted in FIG. 21C. In this variation, the upper voltage connector 2120a may be the positive voltage connector, while the lower connector 2120b may be the negative voltage connector, but in other variations the polarity may be the opposite.

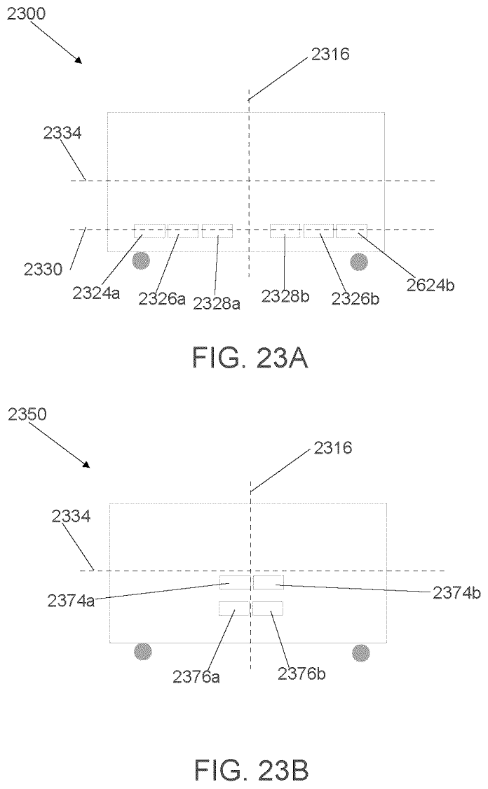

FIGS. 23A and 23B depict additional variations of the connector configuration for the mobile cart 2300 (omitting depiction of the voltage connectors for simplicity). In FIG. 23A, for example, the cart 2300 comprises three pairs of contact connectors 2324a-b, 2326a-b and 2328a-b comprising a midline gap between connector pair 2328a and 2328b, that is larger than the spacing between 2324a and 2326a, between 2326a and 2328a, between 2328b and 2326b, or between 2326b and 2324b, and wherein these gaps between the adjacent connectors on the same side of the midline 2316 have the same interconnector gap spacing, while still all being aligned along a lower horizontal line 2330. The contacts of each connector of each pair are configured to be equidistant from the midline 2316, while also being aligned along a horizontal line 2330 that is lower than the horizontal midline 2334. so that the cart 2300 is usable with different docking or connection orientations. In. FIG. 23B, the cart 2350 is provided with two pairs of connectors 2374a-b and 2376a-b. Both pairs are symmetrical positioned with respect to the vertical midline 2316, but are also spaced apart along the vertical midline 2316, with only the connectors of each pair being horizontally aligned. In this particular example, both pairs 2374a-b and 2376a-b remain below the horizontal midline 2334 of the cart 2350, but in other variations, one or more of the pairs 2374a-b and 2376a-b may be at or above the horizontal midline 2334.

### Cart Rental

The various remote locks described herein for rooms, equipment, drawers and cabinets may be selected to utilize actuators with a locking pin that is extendable and retractable to control its.

### Remote Locks

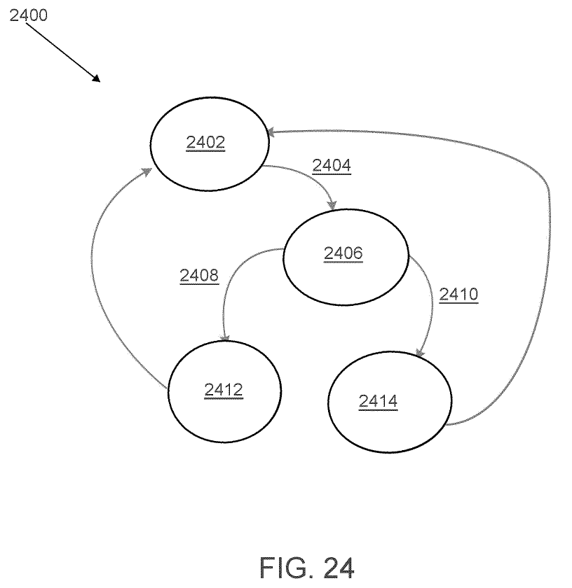

The various remote locks described herein for rooms, equipment, drawers and cabinets may be selected to utilize actuators with a locking pin that is extendable and retractable to control its locking state. Upon the initial application of a voltage (TO), the remote lock actuator will begin to extend its locking pin for a duration (T-EXTEND) until the pin is fully extended. In some variations, continued application of voltage may result in the initiation of pin retraction, with the duration from full extension to full retraction (T-RETRACT). The lock may be further configured so that any further application of voltage will not repeat the extension or retraction phase, and instead keep the locking pin retracted, e.g. the remote lock remains unlocked. Upon detection of the door, drawer or other equipment being opened or otherwise being in use (T-OPEN), The locking pin may enter a catch state in which when the door, drawer or equipment is closed or no longer actively used, the object will relock. To the extend the remote locks and/or remote lock controllers are only in intermittent communication or connectivity, The lock controller may have an accurate signal as to the state of the lock. In some variations, the lock may default to the unlocked state, e.g. to provide resource access unless otherwise instructed. Referring to FIG. 24, to account for potential intermittent signal loss, the state machine 2400 of the lock controller may be configured as follows, one or more processors, and one or more non-transitory computer-readable media storing computer-executable instructions that, when executed, cause the one or more processors to perform actions comprising:
1) An initial State 0 UNKNOWN 2402 where no assumptions are made regarding the lock status of a remote lock.
2) A START sequence 2404 is then initiated, to clear any existing lock actuator operations that were in progress or otherwise loaded.
   a. The remote lock may be powered off for a period of time, e.g. the total of T-EXTEND, T-RETRACT and an offset time, e.g. 1 second to clear the lock state.
3) After the START sequence 2404, the controller moves the remote lock to a State 0 KNOWN 2406, where the remote lock and controller are ready to receive subsequent commands, e.g. a lock command 2408 or a unlock command 2410.
4) During an unlock comment 2410, the remote lock may be powered off.
5) During a lock comment, the remote lock may be powered for a duration T-EXTEND, and then powered off, to put the lock into the locked state.
To help ensure to correct lock state, each machine command ends with the power off, to ensure that any intermittent connectivity does not lock or unlock the cart with a positive intent.

### Network System Structure

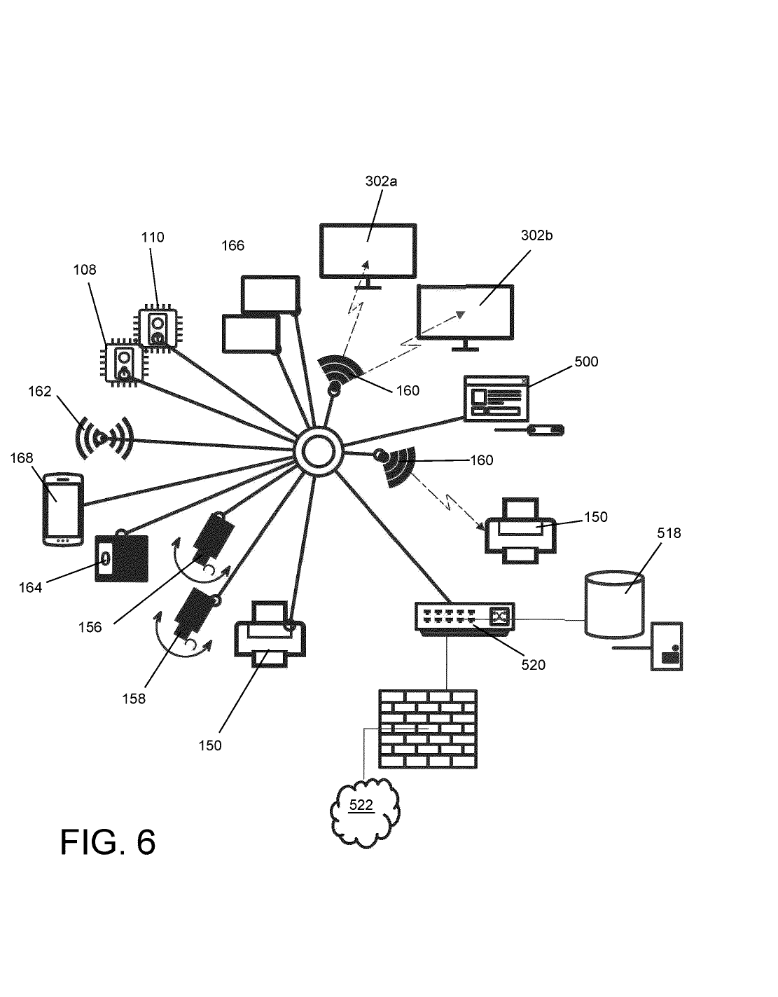

The provisioning module 400 which controls the various locks and other hardware in the co-working facility is part of an orchestration server 500, which is typically located on-site but may also be cloud-based. The orchestration server 500 includes a state machine or management module 502 for physicians (and physician staff), a state machine or management module 504 for patients 504, a display server 506 for managing display output (and optional input) located in the leasable rooms, an application server to interface with onsite physicians and physician staff, patients, as well as onsite or remote facility staff, an optional tracking server 508 configured to track all of the ticket scans and other hardware inputs, a state machine or management module 510 for the various connected hardware and the site management and provisioning module 400 that coordinates the inputs and outputs from the other machines and modules to provide the access control to resources by physicians, staff and patients. This may be performed through a hardware interface 512 that manages wired or wireless one-way and two communications with connected devices 514, 516 respectively. As depicted in FIG. 6, the orchestration server 500 can receive data and control hardware components of the facility through the hardware interface controller 512. The hardware components may include the aforementioned remote-controlled room, cabinet and equipment locks 108, 110, 112 and optionally include auto-clave 140, refrigerator 142, fixed locker(s) 144, mobile cart(s) 146, printer 150 and mailbox 152. In FIG. 6, additional hardware components that may be controlled or that may provide input to the orchestration server 500 may include room or hallway cameras 156, 158, wireless access points 160, RFID readers 162, physical counters 164, facility control screens 166 to control lighting or to perform onsite changes or product ordering, and wired or wireless communications with other mobile devices 168.

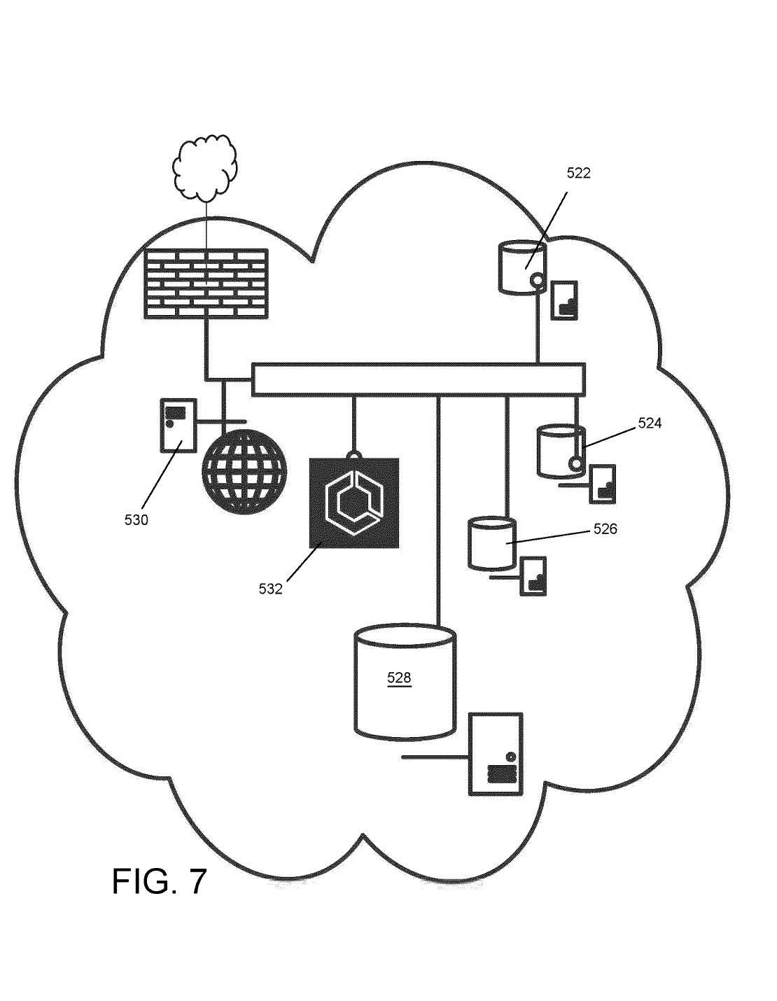

The functions of the orchestration server 500 utilize inputs from the local database 518 via a network interface 520. The network interface 520 also receives data from the any cloud-based servers 522 and services that manage non-local system functions, such as physician account set-up and patient scheduling. For example, the local database 518 in FIG. 6 may be updated and synchronized with a remote or cloud-based site-specific database 522 for that specific facility, depicted schematically in FIG. 7. The remote or cloud-based database 522 may be one of a plurality of cloud-based site-specific databases 524, 526 that are provided for other local facilities that are operating in the same medical co-working system. The data in each of the remote or cloud-based site-specific databases 522, 524, 526 may receive updated data from the local databases as well as filtered or segregated data from a central database 528. The central database 528 may receive data from web servers 530 that provide application services for the physician account module, patient account module, and the like. These databases 522, 524, 526, 528 and application server 530 may be containerized to deployable on any compatible hosting system 532.

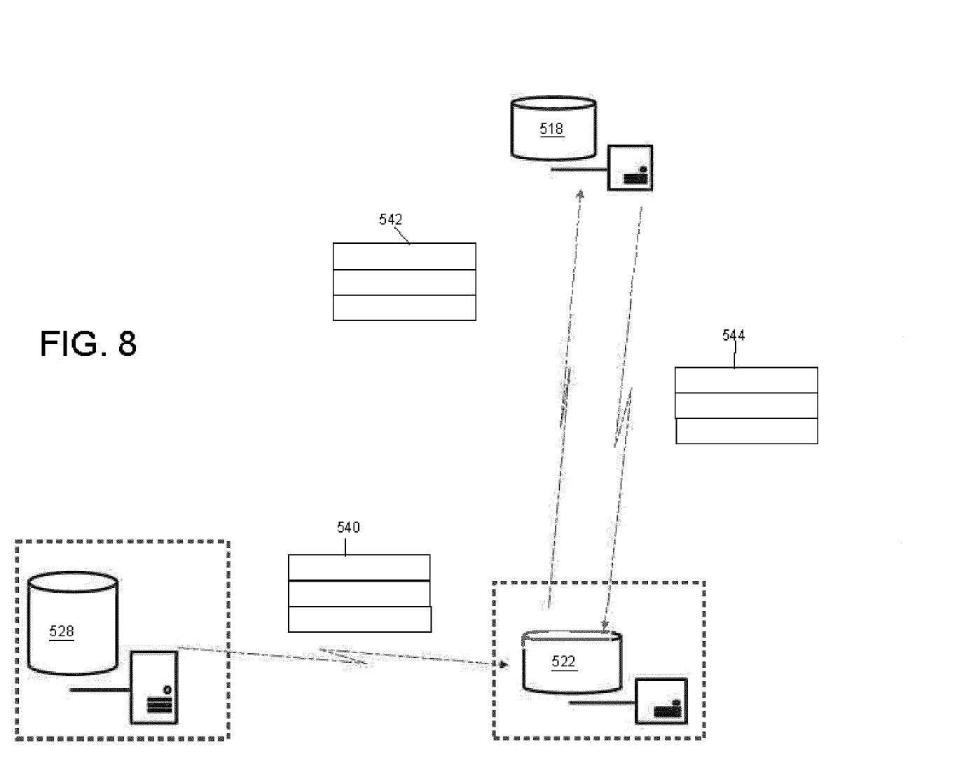

FIG. 8 depicts an exemplary data exchange diagram illustrating the types of data exchanged between a central database and server 528, site-specific remote or cloud database server 522 and a local database 518. Between the central database and server 528 and the remote or cloud database and server 522, there may be various master site configuration tables 540. These may include data structures may be configured or filtered for the specific local site, but otherwise may include but are not limited to facility schedules, room types, hardware types, facility employee types, security attributes, supply types, as well as financial information structures for tax types, general ledger accounts and banking information, for example. Some of the information structures may be hierarchical, nested or tree-based, e.g. security access to a fixed cabinets within an examination or procedure room, or lab equipment in a laboratory, will necessarily require room security. Likewise, patient schedule structures may be located within a physician schedule structure, but patient configuration data structures may be shared across different physician account structures.

From the site-specific remote or cloud database server 522 to the local database 518, various business and site configuration tables or data structures may be transferred. These may include, for example, information related to accounts, leases, employees and security attributes, building schedules, site schedules and inventory types, room and hardware types, site employees and site banking and accounting data structures. Data transfers 544 from the local database 518 to the remote or cloud database and server 522 may include but are not limited to RFID tags for providers, staff, patients, equipment and consumable products; status logs for various remote controller locks and power plugs, room status images or video, people or inventory counters, and room inventory data.

Some variations described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs); Compact Disc-Read Only Memories (CD-ROMs), and holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

In some variations, the systems and methods may be in communication with other computing devices (not shown) via, for example, one or more networks, each of which may be any type of network (e.g., wired network, wireless network). A wireless network may refer to any type of digital network that is not connected by cables of any kind. Examples of wireless communication in a wireless network include, but are not limited to cellular, radio, satellite, and microwave communication. However, a wireless network may connect to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), wireless personal area network (PAN) (e.g., Bluetooth, Bluetooth Low Energy), global area networks (GAN), like the Internet, and virtual private networks (VPN). Hereinafter, network refers to any combination of wireless, wired, public and private data networks that are typically interconnected through the Internet, to provide a unified networking and information access system.

Cellular communication may encompass technologies such as GSM, PCS, CDMA or GPRS, W-CDMA, EDGE or CDMA2000, LTE, WiMAX, and 5G networking standards. Some wireless network deployments combine networks from multiple cellular networks or use a mix of cellular, Wi-Fi, and satellite communication. In some variations, the systems, devices, and methods described herein may include a radiofrequency receiver, transmitter, and/or optical (e.g., infrared) receiver and transmitter to communicate with one or more devices and/or networks.

The specific examples and descriptions herein are exemplary in nature and variations may be developed by those skilled in the art based on the material taught herein without departing from the scope of the present invention, which is limited only by the attached claims.

What is claimed is:

1. A mobile cart system, comprising:
a cart housing;
a plurality of wheels attached to the cart housing;
a plurality of drawers in the cart housing;
a cart door movably attached to the cart housing, the cart door comprising a closed configuration wherein the cart door is located over at least some of the plurality of drawers to resist drawer opening, and an open configuration wherein the cart door is located away from the plurality of drawers to permit drawer opening;
a pair of spring-biased voltage connectors that are either:
vertically aligned along a midline between two halves of the cart housing; or
symmetrically spaced from the midline;
a plurality of pairs of spring-biased surface contact connectors, wherein each pair is symmetrically spaced away from the midline; and
an electronically actuatable door lock connected to at least one pair of the plurality of surface contact connectors.

2. The mobile cart system of claim 1, further comprising a z-clip located symmetrically on the midline.

3. The mobile cart system of claim 1, wherein each pair of spring-biased surface contact connectors comprises spring-biased z-clips.

4. The mobile cart system of claim 1, wherein the pair of spring-biased voltage connectors are symmetrically spaced from the midline.

5. The mobile cart system of claim 4, wherein the pair of spring-biased voltage connectors are spring-biased z-clips.

6. The mobile cart system of claim 5, wherein each pair of the plurality of pairs of spring-biased surface contact connectors comprises spring-biased z-clips.

7. The mobile cart system of claim 4, wherein the pair of spring-biased voltage connectors and the plurality of pairs of spring-biased surface contact connectors are horizontally aligned.

8. The mobile cart system of claim 7, wherein the pair of spring-biased voltage connectors and the plurality of pairs of spring-biased surface contact connectors each comprise z-clips.

9. The mobile cart system of claim 1, further comprising a cart identification tag.

10. The mobile cart system of claim 9, wherein the cart identification tag is located on a rear wall of the cart housing.

11. The mobile cart system of claim 9, wherein the cart identification tag is located symmetrically on the midline.

12. The mobile cart system of claim 11, wherein the cart identification tag is spaced closer to a top edge of the cart housing than to the closest of any pair of the plurality of pairs of spring-biased surface contact connectors.

13. A medical co-working space, comprising:
a reception area;
one or more check-in stations located in the reception area, each check-in station comprising:
a non-contact thermometer assembly configured to measure a patient temperature;
a display;
a patient input device;
a security access dispenser;
a plurality of medical spaces, wherein at least two of the medical spaces are patient examination rooms, each examination room comprising:
a remote room door control lock;
a room door security access sensor;
a patient examination table;
a first display;
a first remote storage compartment control lock;
a first storage compartment;
a ceiling or wall mounted camera;
a scheduling and provisioning server, comprising:
a database of clinician profiles;
a database of patient profiles, wherein each patient profile is associated with a clinician profile in the database of clinician profiles;
a database of clinician medical space reservations, wherein each of the clinician medical space reservations is associated with a clinician profile, at least one patient examination room of the plurality of medical spaces, and comprises a reservation time period;
a communication link to the one or more check-in stations;
a verification module configured to:
receive data from the patient input device, the PPG assembly and the thermometer; and
actuate the security access dispenser based on the received data;
a security controller configured to control the remote room door control locks and the first remote storage compartment control locks based on the reservation time period and the at least one patient examination room associated with each reservation;
a display server configured to display clinician information on the first display of the patient examination room during the corresponding reservation time period of a clinician medical space reservation for one of the patient examination rooms.

14. The co-working space of claim 13, wherein the security access dispenser is a code printer or a security card dispenser.

15. The co-working space of claim 13, wherein the clinician information comprises at least one of a medical school diploma, residency certificate, fellowship certificate and board certification.

16. The co-working space of claim 15, wherein the clinician information is a scanned image of a physical medical school diploma, residency certificate, fellowship certificate or board certification.

17. The co-working space of claim 13, wherein the scheduling and provisioning server is configured to generate a clinician medical space reservation for the database of clinical medical space reservations based on a reservation request associated with a clinician profile in the database of clinician profiles and the existing clinician medical space reservations in the database of clinician medical space reservations.

18. The co-working space of claim 13, wherein the patient examination rooms each further comprise a first storage compartment security access sensor.

19. The co-working space of claim 13, wherein the plurality of medical spaces further comprises at least one waiting room, the at least one waiting room comprising a remote waiting room door lock, a plurality of seats, and a waiting room display.

20. The co-working space of claim 19, wherein the at least one waiting room further comprises a refreshment station.

21. The co-working space of claim 13, wherein the plurality of medical spaces further comprises at least one nursing station.

22. The co-working space of claim 13, wherein the plurality of medical spaces further comprises at least one waiting room.

23. The co-working space of claim 13, wherein the plurality of medical spaces further comprises at least one laboratory room.

24. The co-working space of claim 13, wherein the plurality of medical spaces further comprises at least one patient check-out area.

25. The co-working space of claim 13, wherein the plurality of medical spaces further comprises at least one specimen room, the specimen room comprising a bathroom.

26. The co-working space of claim 25, wherein the at least one specimen room further comprises a specimen cabinet with a door, wherein the specimen cabinet is located in a wall of between the specimen room and a laboratory room.

27. The co-working space of claim 13, wherein each of the remote room door control lock and the first remote storage compartment control lock comprises a visual status indicator configured to facilitate identification of unlocked or unlockable rooms or storage compartments.

28. The co-working space of claim 13, further comprising a plurality of mobile carts, wherein each of the plurality of mobile carts comprises:
a cart housing;
a plurality of wheels attached to the cart housing;
a plurality of drawers in the cart housing;
a cart door movably attached to the cart housing, the cart door comprising a closed configuration wherein the cart door is located over the plurality of drawers to resist drawer opening, and an open configuration wherein the cart door is located away from the plurality of drawers to permit drawer opening;
an electronically actuatable door lock;
a door lock sensor; and
an RFID identifier.

29. The co-working space of claim 28, wherein the cart housing of each of the plurality of mobile carts comprises at least one wall attachment structure, and each examination room of the plurality of examination room comprises at least one cart attachment structure.

30. The co-working space of claim 29, wherein the at least one wall attachment structure and the at least one cart attachment structure comprises complementary Z-clip strips.

31. The co-working space of claim 29, wherein:
the at least one wall attachment structure comprises a plurality of wall attachment structures, and wherein the at least one cart attachment structure comprises a plurality of cart attachment structures; and
wherein the plurality of wall attachment structures and the plurality of cart attachment structures each have an elongate horizontally oriented shape.

32. The co-working space of claim 31, wherein the plurality of wall attachment structures and the plurality of cart attachment structures comprise complementary horizontally staggered or offset arrangement.

33. The co-working space of claim 28, wherein each of the plurality of mobile carts further comprise a door closure sensor.

34. A mobile cart, comprising:
a cart housing;
a plurality of wheels attached to the cart housing;
a plurality of drawers in the cart housing;
a cart door movably attached to the cart housing, the cart door comprising a closed configuration wherein the cart door is located over the plurality of drawers to resist drawer opening, and an open configuration wherein the cart door is located away from the plurality of drawers to permit drawer opening;
a plurality of elongate Z-clips arranged in a horizontal parallel offset configuration on a rear surface of the cart housing;
a plurality of surface contact connectors located on the plurality of elongate-Z-clips; and
an electronically actuatable door lock connected to at least two of the plurality of surface contact connectors.

35. The mobile cart of claim 34, further comprising a door lock sensor connected to at least two of the plurality of surface contact connectors.

36. The mobile cart of claim 34, further comprising a door closure sensor connected to at least two of the plurality of surface contact connectors.
